SEQUENCE LISTING (106 sequences)

SEQ ID NO: 1 - DNA, 95 bp, Artificial Sequence
  MicroRNA library sequence
aatgatacggcgaccaccgacaggttcagagttctacagtccgacgatctgaggtagtaggttgtatagtccaatcgtatgccgtcttctgcttg

SEQ ID NO: 2 - DNA, 96 bp, Artificial Sequence
  MicroRNA library sequence
aatgatacggcgaccaccgacaggttcagagttctacagtccgacgatctgaggtagtaggttgtgtggttgcgctcgtatgccgtcttctgcttg

SEQ ID NO: 3 - DNA, 96 bp, Artificial Sequence
  MicroRNA library sequence
aatgatacggcgaccaccgacaggttcagagttctacagtccgacgatctgaggtagtaggttgtgtggtttagctcgtatgccgtcttctgcttg

SEQ ID NO: 4 - DNA, 96 bp, Artificial Sequence
  MicroRNA library sequence
aatgatacggcgaccaccgacaggttcagagttctacagtccgacgatcaaggaaggcagcaggcgcgcaaatattcgtatgccgtcttctgcttg

SEQ ID NO: 5 - DNA, 96 bp, Artificial Sequence
  MicroRNA library sequence
aatgatacggcgaccaccgacaggttcagagttctacagtccgacgatctgaggtaggaggttgtatagttgcgctcgtatgccgtcttctgcttg

SEQ ID NO: 6 - DNA, 96 bp, Artificial Sequence
  MicroRNA library sequence
aatgatacggcgaccaccgacaggttcagagttctacagtccgacgatctgaggtagtagattgtatagttgcgctcgtatgccgtcttctgcttg

SEQ ID NO: 7 - DNA, 96 bp, Artificial Sequence
  MicroRNA library sequence
aatgatacggcgaccaccgacaggttcagagttctacagtccgacgatcaacccgtagatccgaacttgtggcgctcgtatgccgtcttctgcttg

SEQ ID NO: 8 - DNA, 95 bp, Artificial Sequence
  MicroRNA library sequence
aatgatacggcgaccaccgacaggttcagagttctacagtccgacgatctacagtactgtgataactgaaatattcgtatgccgtcttctgcttg

SEQ ID NO: 9 - DNA, 97 bp, Artificial Sequence
  MicroRNA library sequence
aatgatacggcgaccaccgacaggttcagagttctacagtccgacgatcagcagcattgtacagggctatgatagctcgtatgccgtcttctgcttg

SEQ ID NO: 10 - DNA, 95 bp, Artificial Sequence
  MicroRNA library sequence
aatgatacggcgaccaccgacaggttcagagttctacagtccgacgatctaaagtgctgacagtgcagattagctcgtatgccgtcttctgcttg

SEQ ID NO: 11 - DNA, 94 bp, Artificial Sequence
  MicroRNA library sequence
aatgatacggcgaccaccgacaggttcagagttctacagtccgacgatctaaggcacgcggtgaatgccatattcgtatgccgtcttctgcttg

SEQ ID NO: 12 - DNA, 96 bp, Artificial Sequence
  MicroRNA library sequence
aatgatacggcgaccaccgacaggttcagagttctacagtccgacgatctccctgagaccctttaacctgtgcgctcgtatgccgtcttctgcttg

SEQ ID NO: 13 - DNA, 96 bp, Artificial Sequence
  MicroRNA library sequence
aatgatacggcgaccaccgacaggttcagagttctacagtccgacgatctccctgagaccctaacttgtgaccaatcgtatgccgtcttctgcttg

SEQ ID NO: 14 - DNA, 96 bp, Artificial Sequence
  MicroRNA library sequence
aatgatacggcgaccaccgacaggttcagagttctacagtccgacgatcctgaagctcagagggctctgattagctcgtatgccgtcttctgcttg

SEQ ID NO: 15 - DNA, 96 bp, Artificial Sequence; identical to SEQ ID NO: 14
  MicroRNA library sequence

SEQ ID NO: 16 - DNA, 91 bp, Artificial Sequence
  MicroRNA library sequence
aatgatacggcgaccaccgacaggttcagagttctacagtccgacgatctcccaccgctgccacccgcgctcgtatgccgtcttctgcttg

SEQ ID NO: 17 - DNA, 96 bp, Artificial Sequence
  MicroRNA library sequence
aatgatacggcgaccaccgacaggttcagagttctacagtccgacgatcaagcccttaccccaaaaagtatatattcgtatgccgtcttctgcttg

SEQ ID NO: 18 - DNA, 96 bp, Artificial Sequence
  MicroRNA library sequence
aatgatacggcgaccaccgacaggttcagagttctacagtccgacgatccagtgcaatgttaaaagggcattagctcgtatgccgtcttctgcttg

SEQ ID NO: 19 - DNA, 96 bp, Artificial Sequence
  MicroRNA library sequence
aatgatacggcgaccaccgacaggttcagagttctacagtccgacgatctaacagtctacagccatggtcgccaatcgtatgccgtcttctgcttg

SEQ ID NO: 20 - DNA, 95 bp, Artificial Sequence
  MicroRNA library sequence
aatgatacggcgaccaccgacaggttcagagttctacagtccgacgatccataaagtagaaagcactactccaatcgtatgccgtcttctgcttg

SEQ ID NO: 21 - DNA, 96 bp, Artificial Sequence
  MicroRNA library sequence
aatgatacggcgaccaccgacaggttcagagttctacagtccgacgatctgagatgaagcactgtagctctatattcgtatgccgtcttctgcttg

SEQ ID NO: 22 - DNA, 96 bp, Artificial Sequence
  MicroRNA library sequence
aatgatacggcgaccaccgacaggttcagagttctacagtccgacgatctgccctgtggactcagttctggatattcgtatgccgtcttctgcttg

SEQ ID NO: 23 - DNA, 97 bp, Artificial Sequence
  MicroRNA library sequence
aatgatacggcgaccaccgacaggttcagagttctacagtccgacgatctctggctccgtgtcttcactcccatattcgtatgccgtcttctgcttg

SEQ ID NO: 24 - DNA, 96 bp, Artificial Sequence
  MicroRNA library sequence
aatgatacggcgaccaccgacaggttcagagttctacagtccgacgatctctcccaacccttgtaccagtgtagctcgtatgccgtcttctgcttg

SEQ ID NO: 25 - DNA, 96 bp, Artificial Sequence
  MicroRNA library sequence
aatgatacggcgaccaccgacaggttcagagttctacagtccgacgatctagcagcacataatggtttgtgccaatcgtatgccgtcttctgcttg

SEQ ID NO: 26 - DNA, 96 bp, Artificial Sequence
  MicroRNA library sequence
aatgatacggcgaccaccgacaggttcagagttctacagtccgacgatctagcagcacgtaaatattggcggcgctcgtatgccgtcttctgcttg

SEQ ID NO: 27 - DNA, 97 bp, Artificial Sequence
  MicroRNA library sequence
aatgatacggcgaccaccgacaggttcagagttctacagtccgacgatcaacattcaacgctgtcggtgagttagctcgtatgccgtcttctgcttg

SEQ ID NO: 28 - DNA, 95 bp, Artificial Sequence
  MicroRNA library sequence
aatgatacggcgaccaccgacaggttcagagttctacagtccgacgatcaacattcaacgctgtcggtgaccaatcgtatgccgtcttctgcttg

SEQ ID NO: 29 - DNA, 115 bp, Artificial Sequence
  MicroRNA library sequence
aatgatacggcgaccaccgacaggttcagagttctacagtccgacgatcattgatcatcgacacttcgaacgcacttgcggccccgggttgcgctcgtatgccgtcttctgcttg

SEQ ID NO: 30 - DNA, 116 bp, Artificial Sequence
  MicroRNA library sequence
aatgatacggcgaccaccgacaggttcagagttctacagtccgacgatccattgatcatcgacacttcgaacgcacttgcggccccgggtttagctcgtatgccgtcttctgcttg

SEQ ID NO: 31 - DNA, 96 bp, Artificial Sequence
  MicroRNA library sequence
aatgatacggcgaccaccgacaggttcagagttctacagtccgacgatctggagagaaaggcagttcctgagcgctcgtatgccgtcttctgcttg

SEQ ID NO: 32 - DNA, 96 bp, Artificial Sequence
  MicroRNA library sequence
aatgatacggcgaccaccgacaggttcagagttctacagtccgacgatccaaagaattctccttttgggctatattcgtatgccgtcttctgcttg

SEQ ID NO: 33 - DNA, 96 bp, Artificial Sequence
  MicroRNA library sequence
aatgatacggcgaccaccgacaggttcagagttctacagtccgacgatctcgtgtcttgtgttgcagccggatattcgtatgccgtcttctgcttg

SEQ ID NO: 34 - DNA, 95 bp, Artificial Sequence
  MicroRNA library sequence
aatgatacggcgaccaccgacaggttcagagttctacagtccgacgatcctcccacatgcagggtttgcaatattcgtatgccgtcttctgcttg

SEQ ID NO: 35 - DNA, 95 bp, Artificial Sequence
  MicroRNA library sequence
aatgatacggcgaccaccgacaggttcagagttctacagtccgacgatcctcccacatgcagggtttgcaccaatcgtatgccgtcttctgcttg

SEQ ID NO: 36 - DNA, 97 bp, Artificial Sequence
  MicroRNA library sequence
aatgatacggcgaccaccgacaggttcagagttctacagtccgacgatccaacggaatcccaaaagcagctgccaatcgtatgccgtcttctgcttg

SEQ ID NO: 37 - DNA, 96 bp, Artificial Sequence
  MicroRNA library sequence
aatgatacggcgaccaccgacaggttcagagttctacagtccgacgatctaggtagtttcatgttgttgggatattcgtatgccgtcttctgcttg

SEQ ID NO: 38 - DNA, 94 bp, Artificial Sequence
  MicroRNA library sequence
aatgatacggcgaccaccgacaggttcagagttctacagtccgacgatcagaggtatagggcatgggaatagctcgtatgccgtcttctgcttg

SEQ ID NO: 39 - DNA, 94 bp, Artificial Sequence; identical to SEQ ID NO: 38
  MicroRNA library sequence

SEQ ID NO: 40 - DNA, 96 bp, Artificial Sequence
  MicroRNA library sequence
aatgatacggcgaccaccgacaggttcagagttctacagtccgacgatctccttcattccaccggagtctggcgctcgtatgccgtcttctgcttg

SEQ ID NO: 41 - DNA, 97 bp, Artificial Sequence
  MicroRNA library sequence
aatgatacggcgaccaccgacaggttcagagttctacagtccgacgatctaaagtgcttatagtgcaggtagtagctcgtatgccgtcttctgcttg

SEQ ID NO: 42 - DNA, 97 bp, Artificial Sequence
  MicroRNA library sequence
aatgatacggcgaccaccgacaggttcagagttctacagtccgacgatccaaagtgctcatagtgcaggtaggcgctcgtatgccgtcttctgcttg

SEQ ID NO: 43 - DNA, 95 bp, Artificial Sequence
  MicroRNA library sequence
aatgatacggcgaccaccgacaggttcagagttctacagtccgacgatctaacagtctccagtcacggccatattcgtatgccgtcttctgcttg

SEQ ID NO: 44 - DNA, 93 bp, Artificial Sequence
  MicroRNA library sequence
aatgatacggcgaccaccgacaggttcagagttctacagtccgacgatctcagagttctacagtctgatagctcgtatgccgtcttctgcttg

SEQ ID NO: 45 - DNA, 97 bp, Artificial Sequence
  MicroRNA library sequence
aatgatacggcgaccaccgacaggttcagagttctacagtccgacgatcttgtgcttgatctaaccatgtgaccaatcgtatgccgtcttctgcttg

SEQ ID NO: 46 - DNA, 96 bp, Artificial Sequence
  MicroRNA library sequence
aatgatacggcgaccaccgacaggttcagagttctacagtccgacgatcagaattgtggctggacatctgtatattcgtatgccgtcttctgcttg

SEQ ID NO: 47 - DNA, 96 bp, Artificial Sequence
  MicroRNA library sequence
aatgatacggcgaccaccgacaggttcagagttctacagtccgacgatcaagctgccagttgaagaactgttagctcgtatgccgtcttctgcttg

SEQ ID NO: 48 - DNA, 97 bp, Artificial Sequence
  MicroRNA library sequence
aatgatacggcgaccaccgacaggttcagagttctacagtccgacgatcagctacattgtctgctgggtttctagctcgtatgccgtcttctgcttg

SEQ ID NO: 49 - DNA, 95 bp, Artificial Sequence
  MicroRNA library sequence
aatgatacggcgaccaccgacaggttcagagttctacagtccgacgatcagctacatctggctactgggtatattcgtatgccgtcttctgcttg

SEQ ID NO: 50 - DNA, 95 bp, Artificial Sequence
  MicroRNA library sequence
aatgatacggcgaccaccgacaggttcagagttctacagtccgacgatcatcacattgccagggatttccatattcgtatgccgtcttctgcttg

SEQ ID NO: 51 - DNA, 96 bp, Artificial Sequence
  MicroRNA library sequence
aatgatacggcgaccaccgacaggttcagagttctacagtccgacgatccattgcacttgtctcggtctgatagctcgtatgccgtcttctgcttg

SEQ ID NO: 52 - DNA, 96 bp, Artificial Sequence
  MicroRNA library sequence
aatgatacggcgaccaccgacaggttcagagttctacagtccgacgatcttcaagtaatccaggataggcagcgctcgtatgccgtcttctgcttg

SEQ ID NO: 53 - DNA, 95 bp, Artificial Sequence
  MicroRNA library sequence
aatgatacggcgaccaccgacaggttcagagttctacagtccgacgatccaagtaatccaggataggcttccaatcgtatgccgtcttctgcttg

SEQ ID NO: 54 - DNA, 95 bp, Artificial Sequence
  MicroRNA library sequence
aatgatacggcgaccaccgacaggttcagagttctacagtccgacgatcttcaagtaattcaggataggtgcgctcgtatgccgtcttctgcttg

SEQ ID NO: 55 - DNA, 95 bp, Artificial Sequence
  MicroRNA library sequence
aatgatacggcgaccaccgacaggttcagagttctacagtccgacgatcttcacagtggctaagttctgcccaatcgtatgccgtcttctgcttg

SEQ ID NO: 56 - DNA, 97 bp, Artificial Sequence
  MicroRNA library sequence
aatgatacggcgaccaccgacaggttcagagttctacagtccgacgatctaagtgcttccatgtttgagtgtgcgctcgtatgccgtcttctgcttg

SEQ ID NO: 57 - DNA, 96 bp, Artificial Sequence
  MicroRNA library sequence
aatgatacggcgaccaccgacaggttcagagttctacagtccgacgatcctttcagtcggatgtttgcagcgcgctcgtatgccgtcttctgcttg

SEQ ID NO: 58 - DNA, 96 bp, Artificial Sequence
  MicroRNA library sequence
aatgatacggcgaccaccgacaggttcagagttctacagtccgacgatctgtaaacatcctacactcagctccaatcgtatgccgtcttctgcttg

SEQ ID NO: 59 - DNA, 96 bp, Artificial Sequence
  MicroRNA library sequence
aatgatacggcgaccaccgacaggttcagagttctacagtccgacgatcctggccctctctgcccttccgtgcgctcgtatgccgtcttctgcttg

SEQ ID NO: 60 - DNA, 96 bp, Artificial Sequence
  MicroRNA library sequence
aatgatacggcgaccaccgacaggttcagagttctacagtccgacgatcctcctatatgatgcctttcttctagctcgtatgccgtcttctgcttg

SEQ ID NO: 61 - DNA, 96 bp, Artificial Sequence
  MicroRNA library sequence
aatgatacggcgaccaccgacaggttcagagttctacagtccgacgatcttataaagcaatgagactgatttagctcgtatgccgtcttctgcttg

SEQ ID NO: 62 - DNA, 96 bp, Artificial Sequence
  MicroRNA library sequence
aatgatacggcgaccaccgacaggttcagagttctacagtccgacgatcaattgcactttagcaatggtgaatattcgtatgccgtcttctgcttg

SEQ ID NO: 63 - DNA, 95 bp, Artificial Sequence
  MicroRNA library sequence
aatgatacggcgaccaccgacaggttcagagttctacagtccgacgatcaataatacatggttgatctttgcgctcgtatgccgtcttctgcttg

SEQ ID NO: 64 - DNA, 96 bp, Artificial Sequence
  MicroRNA library sequence
aatgatacggcgaccaccgacaggttcagagttctacagtccgacgatcagatcgaccgtgttatattcgcgcgctcgtatgccgtcttctgcttg

SEQ ID NO: 65 - DNA, 96 bp, Artificial Sequence
  MicroRNA library sequence
aatgatacggcgaccaccgacaggttcagagttctacagtccgacgatcgcctgctggggtggaacctggtccaatcgtatgccgtcttctgcttg

SEQ ID NO: 66 - DNA, 96 bp, Artificial Sequence
  MicroRNA library sequence
aatgatacggcgaccaccgacaggttcagagttctacagtccgacgatcatcatagaggaaaatccatgtttagctcgtatgccgtcttctgcttg

SEQ ID NO: 67 - DNA, 98 bp, Artificial Sequence
  MicroRNA library sequence
aatgatacggcgaccaccgacaggttcagagttctacagtccgacgatcaagctcggtctgaggcccctcagttagctcgtatgccgtcttctgcttg

SEQ ID NO: 68 - DNA, 96 bp, Artificial Sequence
  MicroRNA library sequence
aatgatacggcgaccaccgacaggttcagagttctacagtccgacgatccagcagcaattcatgttttgaaccaatcgtatgccgtcttctgcttg

SEQ ID NO: 69 - DNA, 96 bp, Artificial Sequence
  MicroRNA library sequence
aatgatacggcgaccaccgacaggttcagagttctacagtccgacgatcttttgcgatgtgttcctaatatgcgctcgtatgccgtcttctgcttg

SEQ ID NO: 70 - DNA, 96 bp, Artificial Sequence
  MicroRNA library sequence
aatgatacggcgaccaccgacaggttcagagttctacagtccgacgatcaactgtttgcagaggaaactgaatattcgtatgccgtcttctgcttg

SEQ ID NO: 71 - DNA, 95 bp, Artificial Sequence
  MicroRNA library sequence
aatgatacggcgaccaccgacaggttcagagttctacagtccgacgatctcactcctctcctcccgtcttgcgctcgtatgccgtcttctgcttg

SEQ ID NO: 72 - DNA, 95 bp, Artificial Sequence
  MicroRNA library sequence
aatgatacggcgaccaccgacaggttcagagttctacagtccgacgatccggggcagctcagtacaggattagctcgtatgccgtcttctgcttg

SEQ ID NO: 73 - DNA, 96 bp, Artificial Sequence
  MicroRNA library sequence
aatgatacggcgaccaccgacaggttcagagttctacagtccgacgatcaatcgtacagggtcatccactttagctcgtatgccgtcttctgcttg

SEQ ID NO: 74 - DNA, 96 bp, Artificial Sequence
  MicroRNA library sequence
aatgatacggcgaccaccgacaggttcagagttctacagtccgacgatcaaacaaacatggtgcacttcttccaatcgtatgccgtcttctgcttg

SEQ ID NO: 75 - DNA, 95 bp, Artificial Sequence
  MicroRNA library sequence
aatgatacggcgaccaccgacaggttcagagttctacagtccgacgatccagcagcacactgtggtttgtccaatcgtatgccgtcttctgcttg

SEQ ID NO: 76 - DNA, 96 bp, Artificial Sequence
  MicroRNA library sequence
aatgatacggcgaccaccgacaggttcagagttctacagtccgacgatcaatgcacccgggcaaggattcttagctcgtatgccgtcttctgcttg

SEQ ID NO: 77 - DNA, 96 bp, Artificial Sequence
  MicroRNA library sequence
aatgatacggcgaccaccgacaggttcagagttctacagtccgacgatcaatgcacccgggcaaggattctccaatcgtatgccgtcttctgcttg

SEQ ID NO: 78 - DNA, 97 bp, Artificial Sequence
  MicroRNA library sequence
aatgatacggcgaccaccgacaggttcagagttctacagtccgacgatctagcagcgggaacagttctgcagatattcgtatgccgtcttctgcttg

SEQ ID NO: 79 - DNA, 97 bp, Artificial Sequence
  MicroRNA library sequence
aatgatacggcgaccaccgacaggttcagagttctacagtccgacgatctagcagcgggaacagttctgcagtagctcgtatgccgtcttctgcttg

SEQ ID NO: 80 - DNA, 96 bp, Artificial Sequence
  MicroRNA library sequence
aatgatacggcgaccaccgacaggttcagagttctacagtccgacgatcggagaaattatccttggtgtgtgcgctcgtatgccgtcttctgcttg

SEQ ID NO: 81 - DNA, 97 bp, Artificial Sequence
  MicroRNA library sequence
aatgatacggcgaccaccgacaggttcagagttctacagtccgacgatctggaagactagtgattttgttgttagctcgtatgccgtcttctgcttg

SEQ ID NO: 82 - DNA, 95 bp, Artificial Sequence
  MicroRNA library sequence
aatgatacggcgaccaccgacaggttcagagttctacagtccgacgatcttaatatcggacaaccattgtatattcgtatgccgtcttctgcttg

SEQ ID NO: 83 - DNA, 96 bp, Artificial Sequence
  MicroRNA library sequence
aatgatacggcgaccaccgacaggttcagagttctacagtccgacgatctattgcacttgtcccggcctgttagctcgtatgccgtcttctgcttg

SEQ ID NO: 84 - DNA, 97 bp, Artificial Sequence
  MicroRNA library sequence
aatgatacggcgaccaccgacaggttcagagttctacagtccgacgatccaaagtgctgttcgtgcaggtaggcgctcgtatgccgtcttctgcttg

SEQ ID NO: 85 - DNA, 96 bp, Artificial Sequence
  MicroRNA library sequence
aatgatacggcgaccaccgacaggttcagagttctacagtccgacgatctgaggtagtaagttgtattgttgcgctcgtatgccgtcttctgcttg

SEQ ID NO: 86 - DNA, 96 bp, Artificial Sequence
  MicroRNA library sequence
aatgatacggcgaccaccgacaggttcagagttctacagtccgacgatctgaggtagtaagttgtattgtttagctcgtatgccgtcttctgcttg

SEQ ID NO: 87 - DNA, 96 bp, Artificial Sequence
  MicroRNA library sequence
aatgatacggcgaccaccgacaggttcagagttctacagtccgacgatcaacccgtagatccgatcttgtgccaatcgtatgccgtcttctgcttg

SEQ ID NO: 88 - RNA, 21 nt, Artificial Sequence
  Synthetic microRNA
cucaggauggcggagcggucu

SEQ ID NO: 89 - RNA, 21 nt, Artificial Sequence
  Synthetic microRNA
cucaggauggcggagcggucu

SEQ ID NO: 90 - RNA, 18 nt, Artificial Sequence
  Synthetic microRNA
cucaggauggagcggucu

SEQ ID NO: 91 - DNA, 34 bp, Artificial Sequence
  Synthetic oligonucleotide sequence
agatcggaagagctcgtatgccgtcttctgcttg

SEQ ID NO: 92 - DNA, 34 bp, Artificial Sequence; identical to SEQ ID NO: 91
  Synthetic oligonucleotide sequence

SEQ ID NO: 93 - DNA, 34 bp, Artificial Sequence; identical to SEQ ID NO: 91
  Synthetic oligonucleotide sequence

SEQ ID NO: 94 - DNA, 45 bp, Artificial Sequence
  Synthetic oligonucleotide sequence
caagcagaagacggcatacgagctcttccgatcttatagtgagtc

SEQ ID NO: 95 - DNA, 26 bp, Artificial Sequence
  Synthetic oligonucleotide sequence
gttcagagttctacagtccgacgatc

SEQ ID NO: 96 - RNA, 26 nt, Artificial Sequence
  Synthetic RNA sequence
guucagaguucuacaguccgacgauc

SEQ ID NO: 97 - DNA, 26 bp, Artificial Sequence
  Synthetic DNA/RNA sequence
gttcagagttctacaguccgacgauc

SEQ ID NO: 98 - DNA, 25 bp, Artificial Sequence
  Synthetic oligonucleotide sequence
atattcgtatgccgtcttctgcttg

SEQ ID NO: 99 - DNA, 25 bp, Artificial Sequence
  Synthetic oligonucleotide sequence
gcgctcgtatgccgtcttctgcttg

SEQ ID NO: 100 - DNA, 25 bp, Artificial Sequence
  Synthetic oligonucleotide sequence
tagctcgtatgccgtcttctgcttg

SEQ ID NO: 101 - DNA, 25 bp, Artificial Sequence
  Synthetic oligonucleotide sequence
ccaatcgtatgccgtcttctgcttg

SEQ ID NO: 102 - DNA, 36 bp, Artificial Sequence
  Synthetic oligonucleotide sequence
caagcagaagacggcatacgannnntatagtgagtc

SEQ ID NO: 103 - DNA, 21 bp, Artificial Sequence
  Synthetic oligonucleotide sequence
caagcagaagacggcatacga

SEQ ID NO: 104 - DNA, 44 bp, Artificial Sequence
  Synthetic oligonucleotide sequence
aatgatacggcgaccaccgacaggttcagagttctacagtccga

SEQ ID NO: 105 - DNA, 21 bp, Artificial Sequence
  Synthetic oligonucleotide sequence
caagcagaagacggcatacga

SEQ ID NO: 106 - DNA, 96 bp, Artificial Sequence
  MicroRNA library sequence
aatgatacggcgaccaccgacaggttcagagttctacagtccgacgatctacgtcatcgttgtcatcgtcatagctcgtatgccgtcttctgcttg

(19) United States
(12) Patent Application Publication
(10) Pub. No.: US 2010/0062494 A1
(43) Pub. Date: Mar. 11, 2010

(54) ENZYMATIC OLIGONUCLEOTIDE PRE-ADENYLATION

(75) Inventors: George M. Church, Brookline, MA (US); François Vigneault, Medford, MA (US); Michael Sismour, Cambridge, MA (US)

Correspondence Address: BANNER & WITCOFF, LTD., 28 STATE STREET, SUITE 1800, BOSTON, MA, 02109-1701, US

(73) Assignee: President and Fellows of Harvard College, Cambridge, MA (US)

(21) Appl. No.: 12/533,304

(22) Filed: Jul. 31, 2009

Related U.S. Application Data
(60) Provisional application No. 61/087,252, filed on Aug. 8, 2008.

(51) Int. Cl. C12P 19/34

(52) U.S. Cl. 435/91.2; 435/91.52

(57) ABSTRACT

Methods and compositions for making and using pre-adenylated oligonucleotide sequences are provided.

This application claims priority to U.S. Provisional Patent Application No. 61/087,252, filed on Aug. 8, 2008 and is hereby incorporated herein by reference in its entirety for all purposes.

### STATEMENT OF GOVERNMENT INTERESTS

This invention was made with Government support under HG003170 awarded by the National Institutes of Health. The Government has certain rights in the invention.

### FIELD

The present invention relates to novel methods and compounds for pre-adenylating oligonucleotide sequences.

### BACKGROUND

MicroRNAs (miRNAs) constitute a large family of short, endogenous, 21-23 nucleotide non-coding RNAs that post-transcriptionally repress gene expression by binding to 3′ untranslated regions (UTRs) of target mRNAs in a sequence-specific fashion to impair mRNAs translation and/or stability (for reviews see Ambros (2004) Nature 431(7006):350; Bartel (2004) Cell 116(2):281). They have been implicated in the regulation of multiple cellular pathways such as cellular differentiation, apoptosis and metabolism (reviewed in Ambros and Chen (2007) Development 134(9):1635). While the majority of miRNAs were identified by cDNA cloning or by analysis of computer predictions, currently available strategies developed for the study of miRNAs rely mainly on the detection of previously reported, known and confirmed miRNAs. Therefore, the most powerful approach to identify and quantify expression levels of new miRNAs remains direct cloning and sequencing. To do so, miRNAs need to be extracted from a total RNA sample followed by ligation of 3′ and 5′ single strand oligonucleotide adapters (Lau et al. (2001) Science 294(5543):858; Pfeffer et al. (2005) Curr. Prot. Mol. Biol. Chapter 26, Unit 26:4). Following reverse transcription, one strategy consists of PCR-amplifying the cDNAs using primers specific to the ligated adapters in order to concatemerize, clone and sequence the final product. A second strategy is to subject the cDNAs directly to new generation cyclic array sequencing such as 454, Illumina, AB-SOLiD, Helicos, or Polonator platforms.

MiRNAs are generated by Dicer processing and have 5′ phosphate and 3′ hydroxyl termini. This property, coupled with their short length, poses a significant challenge during ligation-based capture as circularizations of the miRNAs tend to be the dominant product. Many methods have recently been generated for circumventing this obstacle. One method relies on a dephosphorylation step to prevent self-circularization and/or concatemerization of the miRNAs. However, this process also converts partly degraded RNA products into substrates for T4 RNA ligase.

Another strategy relies on the ligation of a pre-5′,5′-adenylated adapter to the 3′ end of the miRNAs using T4 RNA ligase in the absence of ATP. Id. Because the 5′ phosphate on the miRNA cannot be adenylated in the absence of ATP, no miRNA circularization can occur and the dominant reaction product is the desired miRNA-3′ adapter conjugate. A 5′ adapter is then ligated to this miRNA-3′ adapted molecule using T4 RNA ligase in the presence of ATP. Although this ligation reaction is simple, obtaining the pre-adenylated oligonucleotide needed for the method is not. Until recently, it required chemical synthesis of the adenosine 5′-phosphorimidazolide followed by chemical adenylation of the 5′ phosphate of the oligodeoxynucleotide. Id. In addition to not being trivial for most molecular biologists, the published chemical synthesis in this procedure is a slow process and has been reported with only 10% to 20% yields. Id. More recently, pre-adenylated oligonucleotides have become commercially available, but at such a high cost that only four are available, thus limiting the versatility of the technique with high-throughput methods (e.g., those requiring barcoding) which can entail dozens to thousands of codes.

### SUMMARY

The present invention is based in part on the surprising discovery of an economical and facile method for the efficient production of pre-adenylated oligonucleotides (e.g., barcoded oligonucleotides) having any sequence. Pre-adenylated oligonucleotides are particularly useful for methods such as microRNA capture, high-throughput sequencing applications (e.g., multiplex analysis) and the like.

Accordingly, in certain exemplary embodiments, a method of generating a pre-adenylated oligonucleotide is provided. The method includes providing a first oligonucleotide having a 3′ block and a 5′ phosphate, providing a second oligonucleotide that is partially complementary to the first oligonucleotide, allowing the first oligonucleotide and the second oligonucleotide to hybridize to form a duplex, wherein the second oligonucleotide has a 3′ overhang, contacting the duplex with a DNA ligase and ATP, and allowing the ligase to adenylate the first oligonucleotide to form a pre-adenylated oligonucleotide. In certain aspects, the method includes purifying the adenylated oligonucleotide, e.g., by gel electrophoresis or by binding a label (e.g., a label that can further bind to a column and/or a bead (such as a magnetic bead)) that is optionally present on the second oligonucleotide. In certain aspects of the exemplary embodiments described above and below, the ligase is a DNA ligase such as T4 DNA ligase or an RNA ligase such as T4 RNA ligase 1 or T4 RNA ligase 2.

In certain exemplary embodiments, method for retrieving a nucleic acid sequence from a sample (e.g., a biological or synthetic sample, in solution or on an solid array surface) including providing the pre-adenylated oligonucleotide described above is provided. The method includes contacting the pre-adenylated oligonucleotide to a sample in the presence of ligase and in the absence of ATP, allowing the pre-adenylated oligonucleotide to bind the 3′ end of a nucleic acid sequence from the sample to form a ligation product comprising the nucleic acid sequence, and retrieving (e.g., by gel electrophoresis) the ligation product. In certain aspects, the nucleic acid sequence is single stranded DNA, double stranded DNA, single stranded RNA (e.g., microRNA, siRNA, snoRNA or the like), double stranded RNA or a DNA-RNA chimera.

In certain exemplary embodiments, method for amplifying a nucleic acid sequence from a sample (e.g., a biological or synthetic sample, in solution or on an solid array surface) including providing the pre-adenylated oligonucleotide described above is provided. The method includes contacting the adenylated oligonucleotide to a sample in the presence of ligase and in the absence of ATP, allowing the adenylated oligonucleotide to bind the 3′ end of the nucleic acid sequence from the sample to form a first ligation product, providing a second oligonucleotide sequence to the sample in the presence of ligase and ATP, allowing the second oligonucleotide sequence to bind the first ligation product to form a second ligation product, and amplifying the second ligation product.

In certain exemplary embodiments, a method for sequencing a plurality of nucleic acid sequences is provided. The method includes providing a first oligonucleotide having a 3′ block and a 5′ phosphate, providing a second oligonucleotide that is partially complementary to the first oligonucleotide, allowing the first oligonucleotide and the second oligonucleotide to hybridize to form a duplex, wherein the second oligonucleotide has a 3′ overhang, contacting the duplex with a DNA ligase and ATP, and allowing the ligase to adenylate the first oligonucleotide to form a pre-adenylated oligonucleotide. The method further includes contacting the adenylated oligonucleotide to a sample (e.g., a biological or synthetic sample, in solution or on an solid array surface) in the presence of ligase and in the absence of ATP, allowing the adenylated oligonucleotide to bind the 3′ end of the nucleic acid sequence from the sample to form a first ligation product, providing a third oligonucleotide sequence to the sample in the presence of ligase and ATP, allowing the third oligonucleotide sequence to bind the first ligation product to form a second ligation product, repeating the above steps until a plurality of second ligation products are obtained, and sequencing the plurality of second ligation products.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The foregoing and other features and advantages of the present invention will be more fully understood from the following detailed description of illustrative embodiments taken in conjunction with the accompanying drawings in which:

FIG. 1 schematically depicts enzymatic pre-adenylation of degenerate oligonucleotides. The oligonucleotide (red) is 5′ phosphorylated and optionally has a blocking group on its 3′ end. The oligonucleotide is annealed with a longer complementary template. The template also has degenerate bases to allow proper base pairing with the oligonucleotide, and has an optional label. Enzymatic adenylation is performed (e.g., using DNA ligase in the presence of ATP). The lack of ligation substrate terminates the ligation reaction prior to completion of the reaction, resulting in the formation of a pre-adenylated (App) oligonucleotide. The App oligonucleotide can then optionally be purified (e.g., using gel electrophoresis, affinity purification (e.g., biotin-coupled bead capture) or the like). N represents degenerate bases.

FIGS. 2A-2B schematically depict general end capture and multiplex sequencing methods. A) General end capture process. The App oligonucleotide can be provided with ligase in end capture methods in the absence of ATP, thus avoiding byproduct(s) or erroneous ligation reaction(s) that would normally occur when using ligase in the presence of ATP. Ligation of a 5′ adapter allows for subsequent amplification and/or sequencing. B) Multiplex sequencing of barcoded samples. Multiple samples captured using barcoded App can be pooled together in a single reaction and optionally sequenced (e.g., on new generation platforms in a multiplex fashion).

FIGS. 3A-3B depict sequencing by ligation using pre-adenylated, degenerate oligonucleotides. A) Probes for use in sequencing by ligation reactions can be pre-adenylated on their 5′ (e.g., degenerate) end as described further herein. B) The pre-adenylated oligonucleotides anneal to the templates to be sequenced (the templates can optionally be attached to a solid surface as described further herein). Following visualization of the attached bases, the fluorescent group is cleaved, and a second oligonucleotide is annealed and ligated in the absence of ATP in order to detect a second base of the template sequence.

FIGS. 4A-4B depict pre-adenylation of the 3′ adapter oligonucleotide by T4 DNA ligase. A) Reaction converting the donor oligonucleotide (pD) to the pre-adenylated form (AppD) in the presence (+) and absence (−) of T4 DNA ligase. The superposed lane is a mixture of both previous lanes. The 40-mer oligonucleotide complementary template used in the pre-adenylation reaction is indicated. B) Time course analysis of the pre-adenylation reaction depicted in A.

FIGS. 5A-5B depict miRNA capture by ligation. A) Ligation of 3′ pre-adenylated (AppD) or non-adenylated (pD) adapter to the synthetic miRNA using T4 RNA ligase 2 (RNL2) in absence of ATP. Control reactions (lanes 3-5) using T4 RNA ligase 1 with ATP demonstrate self-circularization and concatemerization of the synthetic phosphorylated (pA) and dephosphorylated (A) miRNA. B) Ligation of the 5′ adapter to the miRNA-3′ adapter ligation product. The ligation was conducted on a PAGE purified fragment produced in A) or directly on the reaction mixture without prior PAGE purification.

FIGS. 6A-6D depict optimization of the ligation of the pre-adenylated 3′ adapter (AppD) to the synthetic miRNA using T4 RNA ligase 2 without ATP. The following parameters were assessed: A) Time course analysis of the ligation reaction; B) T4 RNA ligase 2 (RNL2) concentration; C) polyethylene glycol (PEG) concentration; and D) App-3′ adapter to miRNA ratio.

FIG. 7 depicts 5′ adapter ligation of oligonucleotides of various compositions. DNA, RNA and DNA/RNA chimera oligonucleotides were assessed for their efficiency to act as a 5′ adapter in a T4 RNA ligase 1 ligation with ATP to the miRNA-3′ adapter ligation product generated in FIG. 5A.

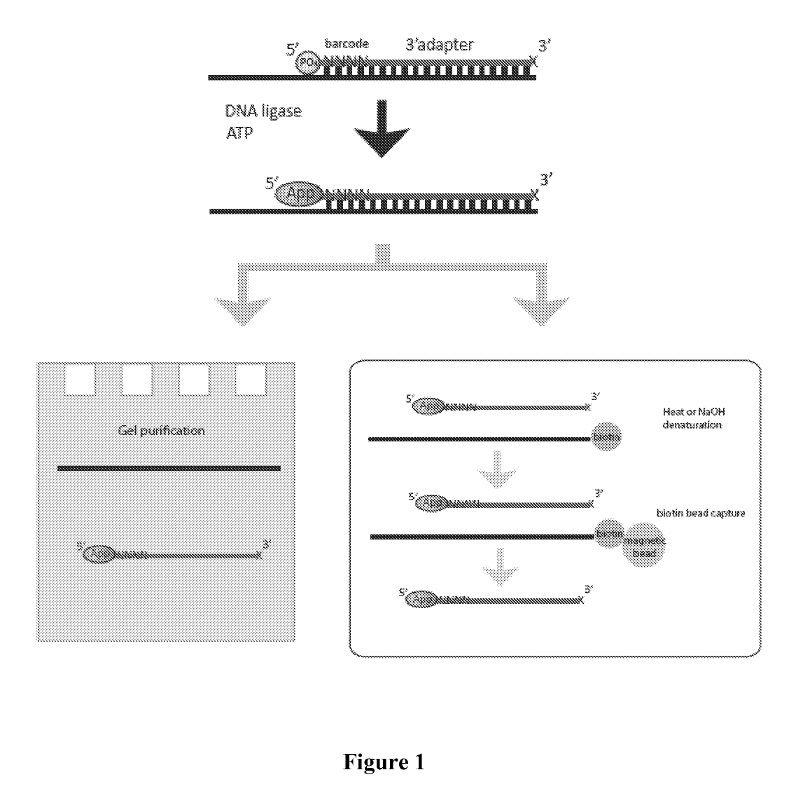

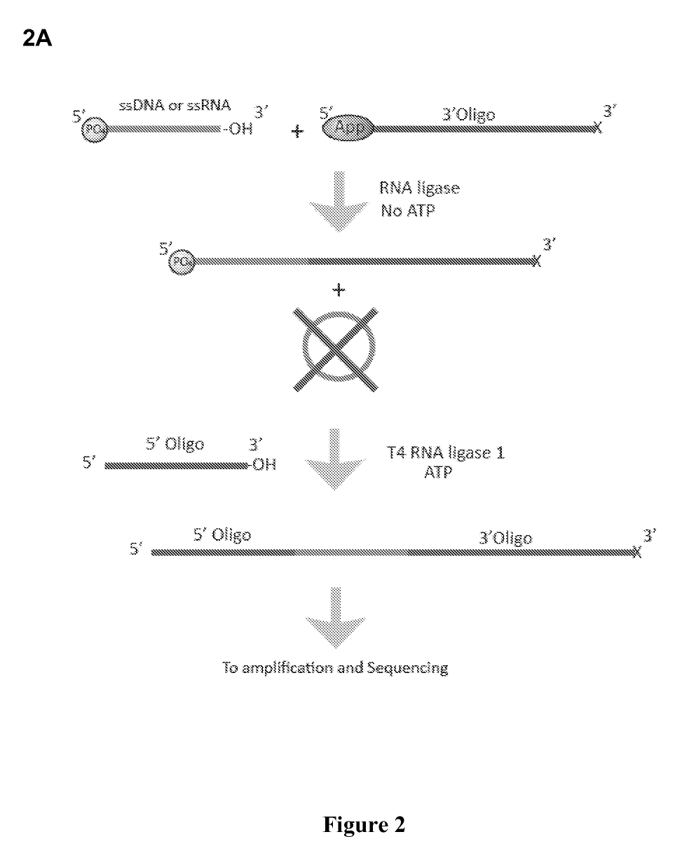

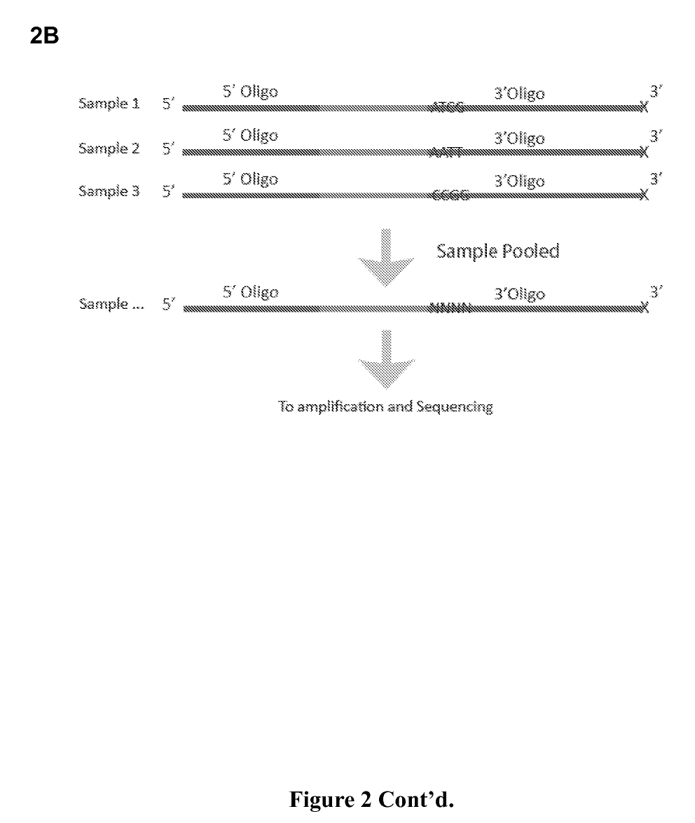

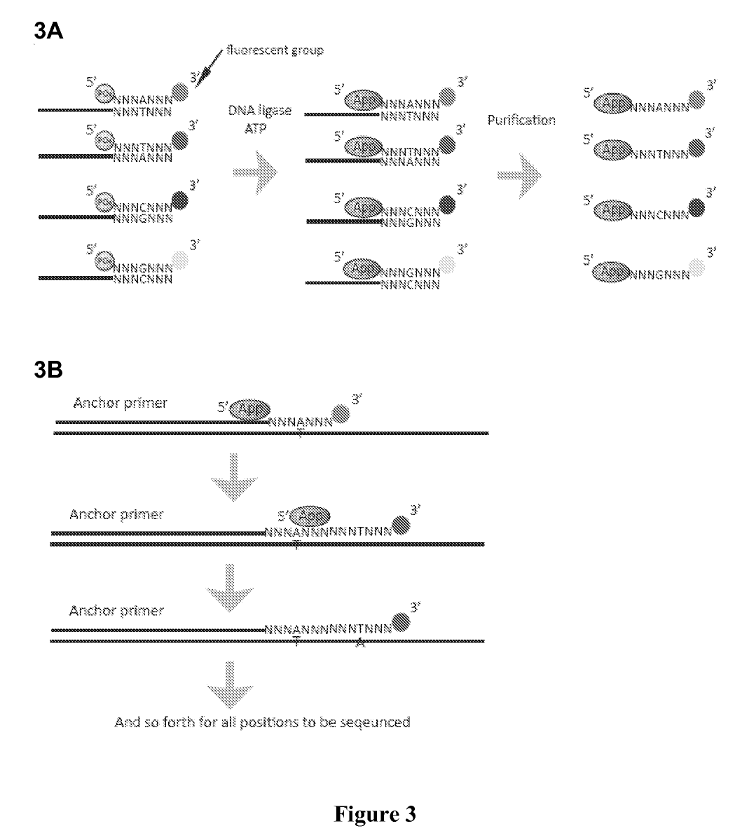

### DETAILED DESCRIPTION

The principles of the present invention may be applied with particular advantage to efficiently and facilely pre-adenylate oligonucleotide sequences. In certain embodiments, adenylation of oligonucleotides (e.g., degenerate oligonucleotides) is achieved by using a complementary template that is longer than the oligonucleotide, and allowing base pair matching of the oligonucleotide to the template. The annealed oligonucleotide with its complementary template is subjected to enzymatic adenylation using ligase (e.g., DNA (e.g., T4)) with ATP. Adenylated oligonucleotides are then purified (e.g., on gels and/or with paramagnetic biotin-beads capture). The adenylated oligonucleotides can then be used in the ligation to any nucleotide substrate having a 3′ hydroxy termini using DNA or RNA ligase without ATP.

The invention provides a highly efficient and simplified strategy to pre-adenylate oligonucleotides (e.g., barcoded oligonucleotides of degenerate sequence) that is useful for a variety of applications such as, e.g., for multiplex barcoding and/or sequencing. Pre-adenylated oligonucleotides can be used in an ATP-independent ligation reactions to covalently link the pre-adenylated oligonucleotide to any substrate composed of nucleic acid, e.g., end capture protocols and high-throughput sequencing applications. The present invention provides novel methods for pre-adenylating any nucleic acid sequence with high efficiency and low cost, and can be applied to any custom nucleotide sequences, such as, e.g., a mixture of degenerate or pooled sequences.

The methods and compositions described herein can be used to produce pre-adenylated single stranded or double stranded DNA, RNA or DNA-RNA chimeric oligonucleotides or adapters. Such pre-adenylated nucleotide sequences can then be used to capture and/or barcode non-exclusively single strand and double stranded DNA or RNA samples by ligation using DNA or RNA ligase without ATP (such as e.g., microRNA, siRNA, snoRNA, ssDNA and the like, or any substrate composed of nucleic acid, from biological or synthetic samples, in solution or on an solid array surface). Subsequently, captured samples can be sequenced or quantitated using a known priming sequence or as an identity tag as described further herein to enable pooling of a large amount of sample in one reaction. Accordingly, the methods and compositions described herein provide tremendous multiplex sequencing capacity, e.g., on cyclic array sequencing using platforms such as Roche 454, Illumina Solexa, AB-SOLiD, Helicos, Polonator platforms and the like. In other exemplary embodiments, methods of making adenylated oligonucleotides for use in sequencing by ligation experiments are provided.

As used herein, a “pre-adenylated oligonucleotide” refers to an oligonucleotide having 5′,5′-adenylate moiety. T4 DNA ligase proceeds by a reaction mechanism that forms 5′,5′-adenylated DNA as an intermediate. In certain exemplary embodiments, a pre-adenylated oligonucleotide is made by incubating one or more oligonucleotides with one or more templates in the presence of a DNA polymerase (e.g., T4 DNA polymerase) and ATP. Without substrate available, DNA polymerase activity is abrogated, resulting in the formation of one or more 5′,5′-adenylated oligonucleotides (i.e., one or more pre-adenylated oligonucleotides).

In certain exemplary embodiments, methods of making pre-adenylated oligonucleotides using one or more ligases are provided. As used herein, the term “ligase” refers to a class of enzymes and their functions in forming a phosphodiester bond in adjacent oligonucleotides which are annealed to the same oligonucleotide. Particularly efficient ligation takes place when the terminal phosphate of one oligonucleotide and the terminal hydroxyl group of an adjacent second oligonucleotide are annealed together across from their complementary sequences within a double helix, i.e. where the ligation process ligates a “nick” at a ligatable nick site and creates a complementary duplex (Blackburn, M. and Gait, M. (1996) in Nucleic Acids in Chemistry and Biology, Oxford University Press, Oxford, pp. 132-33, 481-2). The site between the adjacent oligonucleotides is referred to as the “ligatable nick site,” “nick site,” or “nick,” whereby the phosphodiester bond is non-existent, or cleaved. The term “ligate” refers to the reaction of covalently joining adjacent oligonucleotides through formation of an internucleotide linkage.

Ligases include DNA ligases and RNA ligases. A DNA ligase is an enzyme that closes nicks or discontinuities in one strand of duplex nucleic acids by creating an ester bond between juxtaposed 3′ OH and 5′ PO₄ termini. DNA ligases include, but are not limited to, T4 DNA ligase, Taq DNA ligase, DNA ligase (E. coli) and the like. An RNA ligase is an enzyme that catalyzes ligation of juxtaposed 3′ OH and 5′ PO₄ termini by the formation of a phosphodiester bond. RNA ligases include T4 RNA ligase 1, T4 ligase 2, TS2126 RNA ligase 1 and the like. A variety of ligases are commercially available (e.g., New England Biolabs, Beverly, Mass.).

In certain exemplary embodiments, oligonucleotides may have a blocking group at their 5′ and/or 3′ ends (a 5′ or 3′ block, respectively). For example, a cleavable linker moiety may be covalently attached to the 5′ and/or 3′ ends of oligonucleotides. The linker moiety may be of six or more atoms in length. Alternatively, a cleavable moiety may be within an oligonucleotide and may be introduced during in situ synthesis. A broad variety of cleavable moieties are available in the art of solid phase and microarray oligonucleotide synthesis (see e.g., Pon, R., Methods Mol. Biol. 20:465-496 (1993); Verma et al., Ann. Rev. Biochem. 67:99-134 (1998); U.S. Pat. Nos. 5,739,386, 5,700,642 and 5,830,655; and U.S. Patent Publication Nos. 2003/0186226 and 2004/0106728). Cleavable linkers described in Attorney Docket Number 10498-00190 are also useful for the methods and compositions described herein. The cleavable moiety may be removed under conditions which do not degrade the oligonucleotides.

In certain exemplary embodiments, sequential hybridization is used to determine the presence and/or location of one or more barcode sequences. For example, at each cycle of a sequencing reaction, oligonucleotide sequences complementary to four barcodes, each bearing one of four detectable markers or labels, is hybridized, and images are captured.

As used herein, the term “barcode” refers to a unique oligonucleotide sequence that allows a corresponding nucleic acid base and/or nucleic acid sequence to be identified. In certain aspects, the nucleic acid base and/or nucleic acid sequence is located at a specific position on a larger polynucleotide sequence. In certain embodiments, barcodes can each have a length within a range of from 4 to 36 nucleotides, or from 6 to 30 nucleotides, or from 8 to 20 nucleotides. In certain exemplary embodiments, a barcode has a length of 4 nucleotides. In certain aspects, the melting temperatures of barcodes within a set are within 10° C. of one another, within 5° C. of one another, or within 2° C. of one another. In other aspects, barcodes are members of a minimally cross-hybridizing set. That is, the nucleotide sequence of each member of such a set is sufficiently different from that of every other member of the set that no member can form a stable duplex with the complement of any other member under stringent hybridization conditions. In one aspect, the nucleotide sequence of each member of a minimally cross-hybridizing set differs from those of every other member by at least two nucleotides. Barcode technologies are known in the art and are described in Winzeler et al. (1999) Science 285:901; Brenner (2000) Genome Biol. 1:1 Kumar et al. (2001) Nature Rev. 2:302; Giaever et al. (2004) Proc. Natl. Acad. Sci. USA 101:793; Eason et al. (2004) Proc. Natl. Acad. Sci. USA 101:11046; and Brenner (2004) Genome Biol. 5:240.

As used herein, the terms “nucleic acid molecule,” “nucleic acid sequence,” “nucleic acid fragment,” “oligonucleotide” and “polynucleotide” are used interchangeably and are intended to include, but not limited to, a polymeric form of nucleotides that may have various lengths, either deoxyribonucleotides or ribonucleotides, or analogs thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, ribozymes, small interfering RNA (siRNA), miRNA, small nucleolar RNA (snoRNA), cDNA, recombinant polynucleotides, branched polynucleotides, plasmids, vectors, isolated DNA of a sequence, isolated RNA of a sequence, nucleic acid probes, and primers. Oligonucleotides useful in the methods described herein may comprise natural nucleic acid sequences and variants thereof, artificial nucleic acid sequences, or a combination of such sequences.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

Examples of modified nucleotides include, but are not limited to diaminopurine, S²T, 5-fluorouracil, 5-bromouracil, 5-chlorouracil, 5-iodouracil, hypoxanthine, xantine, 4-acetylcyto sine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-D46-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, 2,6-diaminopurine and the like. Nucleic acid molecules may also be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety or phosphate backbone.

Oligonucleotide sequences may be isolated from natural sources or purchased from commercial sources. Oligonucleotide sequences may also be prepared by any suitable method, e.g., standard phosphoramidite methods such as those described by Beaucage and Carruthers ((1981) Tetrahedron Lett. 22: 1859) or the triester method according to Matteucci et al. (1981) J. Am. Chem. Soc. 103:3185), or by other chemical methods using either a commercial automated oligonucleotide synthesizer or high-throughput, high-density array methods known in the art (see U.S. Pat. Nos. 5,602,244, 5,574,146, 5,554,744, 5,428,148, 5,264,566, 5,141,813, 5,959,463, 4,861,571 and 4,659,774, incorporated herein by reference in its entirety for all purposes). Pre-synthesized oligonucleotides may also be obtained commercially from a variety of vendors.

In certain exemplary embodiments, oligonucleotide sequences may be prepared using a variety of microarray technologies known in the art. Pre-synthesized oligonucleotide and/or polynucleotide sequences may be attached to a support or synthesized in situ using light-directed methods, flow channel and spotting methods, inkjet methods, pin-based methods and bead-based methods set forth in the following references: McGall et al. (1996) Proc. Natl. Acad. Sci. U.S.A. 93:13555; Synthetic DNA Arrays In Genetic Engineering, Vol. 20:111, Plenum Press (1998); Duggan et al. (1999) Nat. Genet. S21:10; Microarrays: Making Them and Using Them In Microarray Bioinformatics, Cambridge University Press, 2003; U.S. Patent Application Publication Nos. 2003/0068633 and 2002/0081582; U.S. Pat. Nos. 6,833,450, 6,830,890, 6,824,866, 6,800,439, 6,375,903 and 5,700,637; and PCT Application Nos. WO 04/031399, WO 04/031351, WO 04/029586, WO 03/100012, WO 03/066212, WO 03/065038, WO 03/064699, WO 03/064027, WO 03/064026, WO 03/046223, WO 03/040410 and WO 02/24597.

In certain exemplary embodiments, one or more oligonucleotide sequences described herein are immobilized on a support (e.g., a solid and/or semi-solid support). In certain aspects, an oligonucleotide sequence can be attached to a support using one or more of the phosphoramidite linkers described herein. Suitable supports include, but are not limited to, slides, beads, chips, particles, strands, gels, sheets, tubing, spheres, containers, capillaries, pads, slices, films, plates and the like. In various embodiments, a solid support may be biological, nonbiological, organic, inorganic, or any combination thereof. When using a support that is substantially planar, the support may be physically separated into regions, for example, with trenches, grooves, wells, or chemical barriers (e.g., hydrophobic coatings, etc.).

In certain exemplary embodiments, a support is a microarray. As used herein, the term “microarray” refers in one embodiment to a type of assay that comprises a solid phase support having a substantially planar surface on which there is an array of spatially defined non-overlapping regions or sites that each contain an immobilized hybridization probe. “Substantially planar” means that features or objects of interest, such as probe sites, on a surface may occupy a volume that extends above or below a surface and whose dimensions are small relative to the dimensions of the surface. For example, beads disposed on the face of a fiber optic bundle create a substantially planar surface of probe sites, or oligonucleotides disposed or synthesized on a porous planar substrate creates a substantially planar surface. Spatially defined sites may additionally be “addressable” in that its location and the identity of the immobilized probe at that location are known or determinable.

Oligonucleotides immobilized on microarrays include nucleic acids that are generated in or from an assay reaction. Typically, the oligonucleotides or polynucleotides on microarrays are single stranded and are covalently attached to the solid phase support, usually by a 5′-end or a 3′-end. In certain exemplary embodiments, probes are immobilized via one or more of the cleavable linkers described herein. The density of non-overlapping regions containing nucleic acids in a microarray is typically greater than 100 per cm², and more typically, greater than 1000 per cm². Microarray technology relating to nucleic acid probes is reviewed in the following exemplary references: Schena, Editor, Microarrays: A Practical Approach (IRL Press, Oxford, 2000); Southern, Current Opin. Chem. Biol., 2: 404-410 (1998); Nature Genetics Supplement, 21:1-60 (1999); and Fodor et al, U.S. Pat. Nos. 5,424,186; 5,445,934; and 5,744,305.

In certain exemplary embodiments, beads are provided for the immobilization of one or more of the oligonucleotides described herein. As used herein, the term “bead” refers to a discrete particle that may be spherical (e.g., microspheres) or have an irregular shape. Beads may be as small as approximately 0.1 μm in diameter or as large approximately several millimeters in diameter. Beads typically range in size from approximately 0.1 μm to 200 μm in diameter. Beads may comprise a variety of materials including, but not limited to, paramagnetic materials, ceramic, plastic, glass, polystyrene, methylstyrene, acrylic polymers, titanium, latex, sepharose, cellulose, nylon and the like.

In accordance with certain embodiments, beads may have functional groups on their surface which can be used to bind nucleic acid sequences to the bead. Nucleic acid sequences can be attached to a bead by hybridization (e.g., binding to a polymer), covalent attachment, magnetic attachment, affinity attachment and the like. For example, the bead can be coated with streptavidin and the nucleic acid sequence can include a biotin moiety. The biotin is capable of binding streptavidin on the bead, thus attaching the nucleic acid sequence to the bead. Beads coated with streptavidin, oligo-dT, and histidine tag binding substrate are commercially available (Dynal Biotech, Brown Deer, WI). Beads may also be functionalized using, for example, solid-phase chemistries known in the art, such as those for generating nucleic acid arrays, such as carboxyl, amino, and hydroxyl groups, or functionalized silicon compounds (see, for example, U.S. Pat. No. 5,919,523).

Methods of immobilizing oligonucleotides to a support are described are known in the art (beads: Dressman et al. (2003) Proc. Natl. Acad. Sci. USA 100:8817, Brenner et al. (2000) Nat. Biotech. 18:630, Albretsen et al. (1990) Anal. Biochem. 189:40, and Lang et al. Nucleic Acids Res. (1988) 16:10861; nitrocellulose: Ranki et al. (1983) Gene 21:77; cellulose: (Goldkorn (1986) Nucleic Acids Res. 14:9171; polystyrene: Ruth et al. (1987) Conference of Therapeutic and Diagnostic Applications of Synthetic Nucleic Acids, Cambridge U.K.; teflon-acrylamide: Duncan et al. (1988) Anal. Biochem. 169:104; polypropylene: Polsky-Cynkin et al. (1985) Clin. Chem. 31:1438; nylon: Van Ness et al. (1991) Nucleic Acids Res. 19:3345; agarose: Polsky-Cynkin et al., Clin. Chem. (1985) 31:1438; and sephacryl: Langdale et al. (1985) Gene 36:201; latex: Wolf et al. (1987) Nucleic Acids Res. 15:2911).

As used herein, the term “attach” refers to both covalent interactions and noncovalent interactions. A covalent interaction is a chemical linkage between two atoms or radicals formed by the sharing of a pair of electrons (i.e., a single bond), two pairs of electrons (i.e., a double bond) or three pairs of electrons (i.e., a triple bond). Covalent interactions are also known in the art as electron pair interactions or electron pair bonds. Noncovalent interactions include, but are not limited to, van der Waals interactions, hydrogen bonds, weak chemical bonds (i.e., via short-range noncovalent forces), hydrophobic interactions, ionic bonds and the like. A review of noncovalent interactions can be found in Alberts et al., in Molecular Biology of the Cell, 3d edition, Garland Publishing, 1994.

In various embodiments, the methods disclosed herein comprise amplification of oligonucleotides. Amplification methods may comprise contacting a nucleic acid with one or more primers that specifically hybridize to the nucleic acid under conditions that facilitate hybridization and chain extension. Exemplary methods for amplifying nucleic acids include the polymerase chain reaction (PCR) (see, e.g., Mullis et al. (1986) Cold Spring Harb. Symp. Quant. Biol. 51 Pt 1:263 and Cleary et al. (2004) Nature Methods 1:241; and U.S. Pat. Nos. 4,683,195 and 4,683,202), anchor PCR, RACE PCR, ligation chain reaction (LCR) (see, e.g., Landegran et al. (1988) Science 241:1077-1080; and Nakazawa et al. (1994) Proc. Natl. Acad. Sci. U.S.A. 91:360-364), self sustained sequence replication (Guatelli et al. (1990) Proc. Natl. Acad. Sci. U.S.A. 87:1874), transcriptional amplification system (Kwoh et al. (1989) Proc. Natl. Acad. Sci. U.S.A. 86:1173), Q-Beta Replicase (Lizardi et al. (1988) BioTechnology 6:1197), recursive PCR (Jaffe et al. (2000) J. Biol. Chem. 275:2619; and Williams et al. (2002) J. Biol. Chem. 277:7790), the amplification methods described in U.S. Pat. Nos. 6,391,544, 6,365,375, 6,294,323, 6,261,797, 6,124,090 and 5,612,199, or any other nucleic acid amplification method using techniques well known to those of skill in the art.

In certain embodiments, methods of determining the nucleic acid sequence of one or more oligonucleotides (e.g., reference oligonucleotides) are provided. Determination of the nucleic acid sequence of a clonally amplified concatemer can be performed using variety of sequencing methods known in the art including, but not limited to, sequencing by hybridization (SBH), sequencing by ligation (SBL), quantitative incremental fluorescent nucleotide addition sequencing (QIFNAS), stepwise ligation and cleavage, fluorescence resonance energy transfer (FRET), molecular beacons, TaqMan reporter probe digestion, pyrosequencing, fluorescent in situ sequencing (FISSEQ), allele-specific oligo ligation assays (e.g., oligo ligation assay (OLA), single template molecule OLA using a ligated linear probe and a rolling circle amplification (RCA) readout, ligated padlock probes, and/or single template molecule OLA using a ligated circular padlock probe and a rolling circle amplification (RCA) readout) and the like. A variety of light-based sequencing technologies are known in the art (Landegren et al. (1998) Genome Res. 8:769-76; Kwok (2000) Pharmocogenomics 1:95-100; and Shi (2001) Clin. Chem. 47:164-172).

In certain exemplary embodiments, methods of multiplex amplification are provided. Methods for multiplexing include, PCR-based assembly methods, e.g., polymerase assembly multiplexing (PAM) described in Tian et al. (2004) Nature 432:1050; incorporated by reference herein in its entirety for all purposes, or ligation based assembly methods (e.g., joining of polynucleotide segments having cohesive ends). In an exemplary embodiment, a plurality of polynucleotide constructs may be assembled in a single reaction mixture. In other embodiments, hierarchical based assembly methods may be used, for example, when synthesizing a large number of polynucleotide constructs, when synthesizing a polynucleotide construct that contains a region of internal homology, or when synthesizing two or more polynucleotide constructs that are highly homologous or contain regions of homology.

In one embodiment, assembly PCR may be used in accordance with the methods described herein. Methods for performing assembly PCR are described, for example, in Kodumal et al. (2004) Proc. Natl. Acad. Sci. U.S.A. 101:15573; Stemmer et al. (1995) Gene 164:49; Dillon et al. (1990) BioTechniques 9:298; Hayashi et al. (1994) BioTechniques 17:310; Chen et al. (1994) J. Am. Chem. Soc. 116:8799; Prodromou et al. (1992) Protein Eng. 5:827; U.S. Pat. Nos. 5,928,905 and 5,834,252; and U.S. Patent Application Publication Nos. 2003/0068643 and 2003/0186226.

In an exemplary embodiment, polymerase assembly multiplexing (PAM) may be used to assemble polynucleotide constructs in accordance with the methods described herein (see e.g., Tian et al. (2004) Nature 432:1050; Zhou et al. (2004) Nucleic Acids Res. 32:5409; and Richmond et al. (2004) Nucleic Acids Res. 32:5011). Polymerase assembly multiplexing involves mixing sets of overlapping oligonucleotides and/or amplification primers under conditions that favor sequence-specific hybridization and chain extension by polymerase using the hybridizing strand as a template. The double stranded extension products may optionally be denatured and used for further rounds of assembly until a desired polynucleotide construct has been synthesized.

In certain exemplary embodiments, a detectable label can be used to detect one or more oligonucleotides described herein. Examples of detectable markers include various radioactive moieties, enzymes, prosthetic groups, fluorescent markers, luminescent markers, bioluminescent markers, metal particles, protein-protein binding pairs, protein-antibody binding pairs and the like. Examples of fluorescent proteins include, but are not limited to, yellow fluorescent protein (YFP), green fluorescence protein (GFP), cyan fluorescence protein (CFP), umbelliferone, fluorescein, fluorescein isothiocyanate, rhodamine, dichlorotriazinylamine fluorescein, dansyl chloride, phycoerythrin and the like. Examples of bioluminescent markers include, but are not limited to, luciferase (e.g., bacterial, firefly, click beetle and the like), luciferin, aequorin and the like. Examples of enzyme systems having visually detectable signals include, but are not limited to, galactosidases, glucorimidases, phosphatases, peroxidases, cholinesterases and the like. Identifiable markers also include radioactive compounds such as ¹²⁵I, ³⁵S, ¹⁴C, or ³H. Identifiable markers are commercially available from a variety of sources.

Fluorescent labels and their attachment to nucleotides and/or oligonucleotides are described in many reviews, including Haugland, Handbook of Fluorescent Probes and Research Chemicals, Ninth Edition (Molecular Probes, Inc., Eugene, 2002); Keller and Manak, DNA Probes, 2nd Edition (Stockton Press, New York, 1993); Eckstein, editor, Oligonucleotides and Analogues: A Practical Approach (IRL Press, Oxford, 1991); and Wetmur, Critical Reviews in Biochemistry and Molecular Biology, 26:227-259 (1991). Particular methodologies applicable to the invention are disclosed in the following sample of references: U.S. Pat. Nos. 4,757,141, 5,151,507 and 5,091,519. In one aspect, one or more fluorescent dyes are used as labels for labeled target sequences, e.g., as disclosed by U.S. Pat. Nos. 5,188,934 (4,7-dichlorofluorescein dyes); 5,366,860 (spectrally resolvable rhodamine dyes); 5,847,162 (4,7-dichlororhodamine dyes); 4,318,846 (ether-substituted fluorescein dyes); 5,800,996 (energy transfer dyes); Lee et al.; 5,066,580 (xanthine dyes); 5,688,648 (energy transfer dyes); and the like. Labelling can also be carried out with quantum dots, as disclosed in the following patents and patent publications: U.S. Pat. Nos. 6,322,901, 6,576,291, 6,423,551, 6,251,303, 6,319,426, 6,426,513, 6,444,143, 5,990,479, 6,207,392, 2002/0045045 and 2003/0017264. As used herein, the term “fluorescent label” includes a signaling moiety that conveys information through the fluorescent absorption and/or emission properties of one or more molecules. Such fluorescent properties include fluorescence intensity, fluorescence lifetime, emission spectrum characteristics, energy transfer, and the like.

Commercially available fluorescent nucleotide analogues readily incorporated into nucleotide and/or oligonucleotide sequences include, but are not limited to, Cy3-dCTP, Cy3-dUTP, Cy5-dCTP, Cy5-dUTP (Amersham Biosciences, Piscataway, N.J.), fluorescein-12-dUTP, tetramethylrhodamine-6-dUTP, TEXAS RED™-5-dUTP, CASCADE BLUE™-7-dUTP, BODIPY TMFL-14-dUTP, BODIPY TMR-14-dUTP, BODIPY TMTR-14-dUTP, RHODAMINE GREEN™-5-dUTP, OREGON GREENR™ 488-5-dUTP, TEXAS RED™-12-dUTP, BODIPY TM 630/650-14-dUTP, BODIPY TM 650/665-14-dUTP, ALEXA FLUOR™ 488-5-dUTP, ALEXA FLUOR™ 532-5-dUTP, ALEXA FLUOR™ 568-5-dUTP, ALEXA FLUOR™ 594-5-dUTP, ALEXA FLUOR™ 546-14-dUTP, fluorescein-12-UTP, tetramethylrhodamine-6-UTP, TEXAS RED™-5-UTP, mCherry, CASCADE BLUE™-7-UTP, BODIPY TM FL-14-UTP, BODIPY TMR-14-UTP, BODIPY TM TR-14-UTP, RHODAMINE GREEN™-5-UTP, ALEXA FLUOR™ 488-5-UTP, LEXA FLUOR™ 546-14-UTP (Molecular Probes, Inc. Eugene, Oreg.) and the like. Protocols are known in the art for custom synthesis of nucleotides having other fluorophores (See, Henegariu et al. (2000) Nature Biotechnol. 18:345).

Other fluorophores available for post-synthetic attachment include, but are not limited to, ALEXA FLUOR™ 350, ALEXA FLUOR™ 532, ALEXA FLUOR™ 546, ALEXA FLUOR™ 568, ALEXA FLUOR™ 594, ALEXA FLUOR™ 647, BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethyl rhodamine, Texas Red (available from Molecular Probes, Inc., Eugene, Oreg.), Cy2, Cy3.5, Cy5.5, Cy7 (Amersham Biosciences, Piscataway, N.J.) and the like. FRET tandem fluorophores may also be used, including, but not limited to, PerCP-Cy5.5, PE-Cy5, PE-Cy5.5, PE-Cy7, PE-Texas Red, APC-Cy7, PE-Alexa dyes (610, 647, 680), APC-Alexa dyes and the like.

Metallic silver or gold particles may be used to enhance signal from fluorescently labeled nucleotide and/or oligonucleotide sequences (Lakowicz et al. (2003) Bio Techniques 34:62).

Biotin, or a derivative thereof, may also be used as a label on an oligonucleotide sequence, and subsequently bound by a detectably labeled avidin/streptavidin derivative (e.g. phycoerythrin-conjugated streptavidin), or a detectably labeled anti-biotin antibody. Digoxigenin may be incorporated as a label and subsequently bound by a detectably labeled anti-digoxigenin antibody (e.g. fluoresceinated anti-digoxigenin). An aminoallyl-dUTP residue may be incorporated into an oligonucleotide sequence and subsequently coupled to an N-hydroxy succinimide (NHS) derivatized fluorescent dye. In general, any member of a conjugate pair may be incorporated into a detection oligonucleotide provided that a detectably labeled conjugate partner can be bound to permit detection. As used herein, the term antibody refers to an antibody molecule of any class, or any sub-fragment thereof, such as an Fab.

Other suitable labels for an oligonucleotide sequence may include fluorescein (FAM), digoxigenin, dinitrophenol (DNP), dansyl, biotin, bromodeoxyuridine (BrdU), hexahistidine (6×His), phosphor-amino acids (e.g. P-tyr, P-ser, P-thr) and the like. In one embodiment the following hapten/antibody pairs are used for detection, in which each of the antibodies is derivatized with a detectable label: biotin/α-biotin, digoxigenin/α-digoxigenin, dinitrophenol (DNP)/α-DNP, 5-Carboxyfluorescein (FAM)/α-FAM.

Oligonucleotide sequences can be indirectly labeled, especially with a hapten that is then bound by a capture agent, e.g., as disclosed in Holtke et al., U.S. Pat. Nos. 5,344,757; 5,702,888; and 5,354,657; Huber et al., U.S. Pat. No. 5,198,537; Miyoshi, U.S. Pat. No. 4,849,336; Misiura and Gait, PCT publication WO 91/17160; and the like. Many different hapten-capture agent pairs are available for use with the invention, either with a target sequence or with a detection oligonucleotide used with a target sequence, as described below. Exemplary, haptens include, biotin, des-biotin and other derivatives, dinitrophenol, dansyl, fluorescein, CY5, and other dyes, digoxigenin, and the like. For biotin, a capture agent may be avidin, streptavidin, or antibodies. Antibodies may be used as capture agents for the other haptens (many dye-antibody pairs being commercially available, e.g., Molecular Probes, Eugene, Oreg.).

In certain exemplary embodiments, a first (e.g., probe) oligonucleotide sequence is annealed to a second (e.g., reference) oligonucleotide sequence. The terms “annealing” and “hybridization,” as used herein, are used interchangeably to mean the formation of a stable duplex. In one aspect, stable duplex means that a duplex structure is not destroyed by a stringent wash, e.g., conditions including temperature of about 5° C. less that the Tₘ of a strand of the duplex and low monovalent salt concentration, e.g., less than 0.2 M, or less than 0.1 M. The term “perfectly matched,” when used in reference to a duplex means that the polynucleotide and/or oligonucleotide strands making up the duplex form a double stranded structure with one another such that every nucleotide in each strand undergoes Watson-Crick base pairing with a nucleotide in the other strand. The term “duplex” includes, but is not limited to, the pairing of nucleoside analogs, such as deoxyinosine, nucleosides with 2-aminopurine bases, PNAs, and the like, that may be employed. A “mismatch” in a duplex between two oligonucleotides means that a pair of nucleotides in the duplex fails to undergo Watson-Crick bonding.

As used herein, the term “hybridization conditions,” will typically include salt concentrations of less than about 1 M, more usually less than about 500 mM and even more usually less than about 200 mM. Hybridization temperatures can be as low as 5° C., but are typically greater than 22° C., more typically greater than about 30° C., and often in excess of about 37° C. Hybridizations are usually performed under stringent conditions, i.e., conditions under which a probe will specifically hybridize to its target subsequence. Stringent conditions are sequence-dependent and are different in different circumstances. Longer fragments may require higher hybridization temperatures for specific hybridization. As other factors may affect the stringency of hybridization, including base composition and length of the complementary strands, presence of organic solvents and extent of base mismatching, the combination of parameters is more important than the absolute measure of any one alone.

Generally, stringent conditions are selected to be about 5° C. lower than the Tₘ for the specific sequence at a defined ionic strength and pH. Exemplary stringent conditions include salt concentration of at least 0.01 M to no more than 1 M Na ion concentration (or other salts) at a pH 7.0 to 8.3 and a temperature of at least 25° C. For example, conditions of 5×SSPE (750 mM NaCl, 50 mM Na phosphate, 5 mM EDTA, pH 7.4) and a temperature of 25-30° C. are suitable for allele-specific probe hybridizations. For stringent conditions, see for example, Sambrook, Fritsche and Maniatis, Molecular Cloning A Laboratory Manual, 2nd Ed. Cold Spring Harbor Press (1989) and Anderson Nucleic Acid Hybridization, 1^{st} Ed., BIOS Scientific Publishers Limited (1999). As used herein, the terms “hybridizing specifically to” or “specifically hybridizing to” or similar terms refer to the binding, duplexing, or hybridizing of a molecule substantially to a particular nucleotide sequence or sequences under stringent conditions.

As used herein, the term “hybridization-based assay” is intended to refer to an assay that relies on the formation of a stable complex as the result of a specific binding event. In one aspect, a hybridization-based assay means any assay that relies on the formation of a stable duplex or triplex between a probe and a target nucleotide sequence for detecting or measuring such a sequence. A “probe” in reference to a hybridization-based assay refers to an oligonucleotide sequence that has a sequence that is capable of forming a stable hybrid (or triplex) with its complement in a target nucleic acid and that is capable of being detected, either directly or indirectly.

The following examples are set forth as being representative of the present invention. These examples are not to be construed as limiting the scope of the invention as these and other equivalent embodiments will be apparent in view of the present disclosure, figures, tables, and accompanying claims. The contents of all references, patents and published patent applications cited throughout this application are hereby incorporated by reference in their entirety for all purposes.

### Example I

### Enzymatic Oligonucleotide Pre-Adenylation

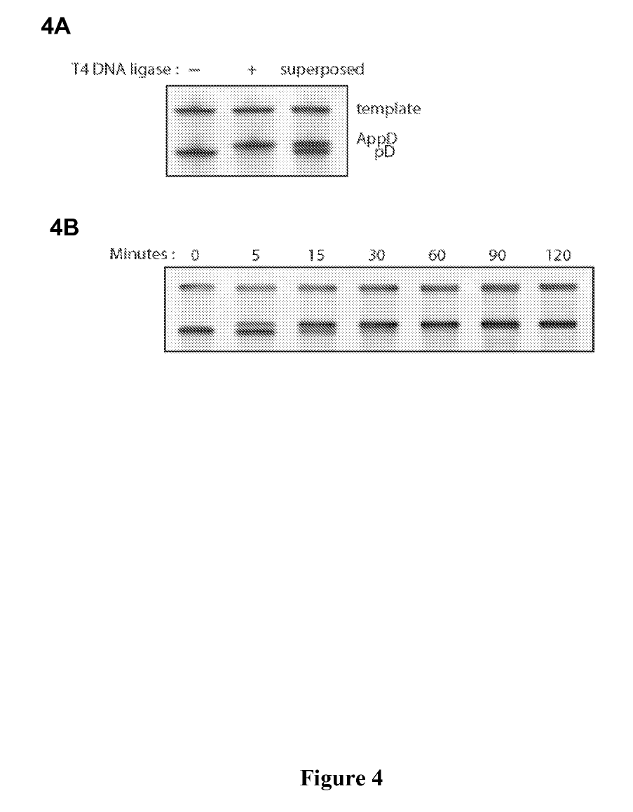

A 34-mer oligonucleotide phosphorylated at the 5′ end and blocked with a 3′-amino modifier (referred as “pD” for 5′ phosphorylated-donor) was generated. Blocking the 3′ termini was critical to avoid self-circularization or ligation to the 3′ end in subsequent steps of miRNA capture. The pD oligonucleotide was first annealed to a 45 nucleotide long complementary oligonucleotide (referred to as the “template”) in such way to create an 11 nucleotide long 3′ overhang of the template oligonucleotide. The annealed oligonucleotide was then incubated with T4 DNA ligase and ATP overnight and analyzed the next day by denaturing PAGE. A clear shift in the migration of the oligonucleotide was observed, indicating the successful addition of a 5′,5′-adenyl pyrophosphoryl cap structure (App) by the T4 DNA ligase interrupted reaction (FIG. 4A). A time course experiment revealed that conversion of the pD oligonucleotide to product was completed after 90 minutes (FIG. 4B). The proportion of non-adenylated pD oligonucleotide was insignificant, allowing facile gel purification of the desired product. Alternatively, the oligonucleotide can be purified using various capture methods such as biotin capture (e.g., via beads), affinity chromatography or the like.

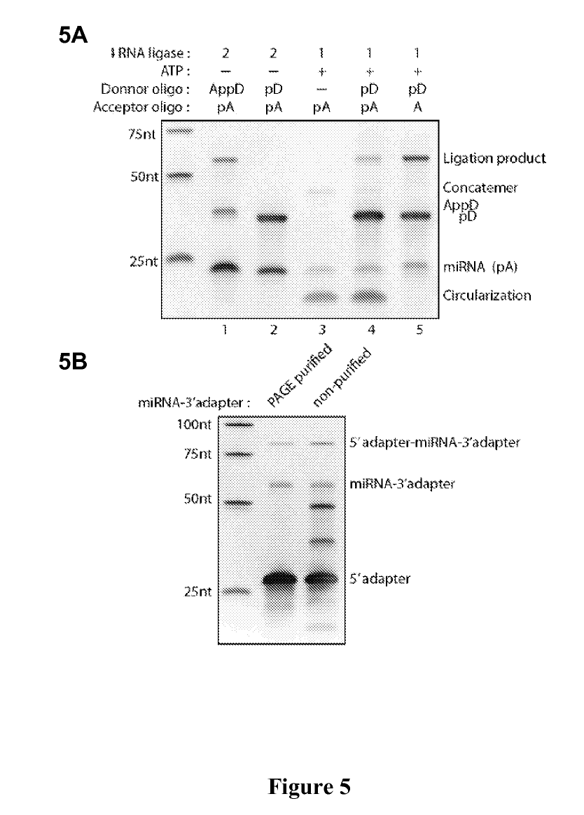

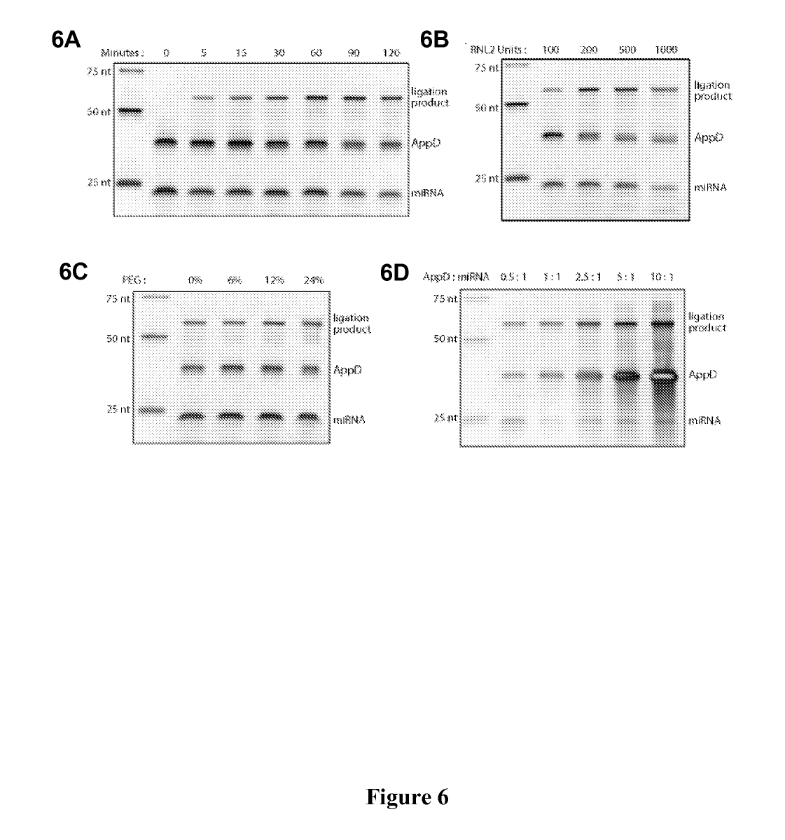

In order to confirm that the AppD was pre-adenylated on its 5′ termini, its efficiency in ligating to the 3′ end of a miRNA was tested. To monitor the reaction, a synthetic 21-mer RNA oligonucleotide 5′ phosphorylated with 3′ hydroxyl termini was used to mimic an actual miRNA (referred as “pA” for 5′-phosphorylated-acceptor or “A” when dephosphorylated). Using T4 RNA ligase 2 (RNL2) without ATP, formation of a 55 nucleotide long ligation product resulting from ligation of the synthetic miRNA and the AppD was observed (FIG. 5A lane 1). This ligation was specific to the pre-adenylated donor oligonucleotide since using RNL2 with the non-adenylated pD did not result in formation of the ligation product (lane 2). As expected, using T4 RNA ligase 1 with ATP allowed for ligation of the non-adenylated form, but resulted in a significant reduction in the ligation due to self-circularization and concatemerization of the synthetic miRNA. While dephosphorylating the miRNA showed strong ligation efficiency (lane 5), it remains a poor strategy if one wants to avoid ligating partially degraded RNA fragments. Since the objective was to achieve maximum capture of miRNA, the efficiency of 3′ adapter ligation was analyzed by testing key variables of this reaction (FIG. 6). It was observed that a reaction time of 60 minutes was sufficient to achieve maximum ligation, while 200 units of RNL2, 12% polyethylene glycol (PEG) and a ratio of 10 to 1 (3′ adapter to miRNA) proved to be optimal. Comparable results were achieved using oligonucleotides of various sequences and sizes (Patel et al. (2008) Bioorg. Chem. 36(2):46). Altogether, these results confirmed the efficiency of the method described herein for producing a pre-adenylated oligonucleotide suitable for miRNA capture by ligation.

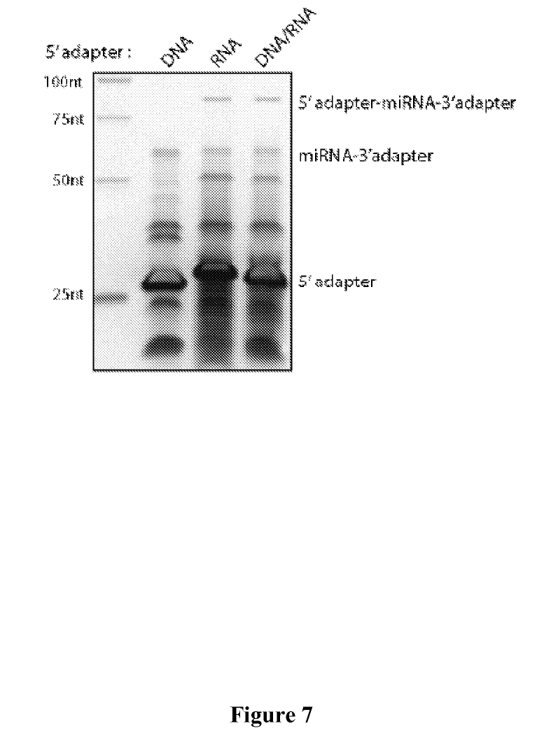

This first ligation was followed by the ligation of a 5′ end adapter (a 26-mer DNA/RNA chimera oligonucleotide). It was observed that skipping gel purification of the initially ligated product resulted in a higher yield of the final 5′ adapter-miRNA-3′ adapter (FIG. 5B). 5′ adapters of different composition (DNA, RNA or DNA/RNA chimeras) were tested and it was observed that the RNA and chimera adapters successfully ligated to the 5′ end of the synthetic miRNA, while the DNA oligonucleotide was unable to achieve such ligation (FIG. 7). Another approach known as 5′-ligation-independent cloning (Pak and Fire (2007) Science 315(5809):241) which uses a second pre-adenylated 5′ adapter on the reverse transcribed strand instead of direct 5′ adapter ligation to the miRNA, will also greatly benefit from simple pre-adenylation of oligonucleotides described herein.

### Example II

### Enzymatic Oligonucleotide Pre-Adenylation and Multiplexing

While 678 miRNAs have been reported to be expressed in human cells (mirBase 11.0, Worldwide Website: microrna.sanger.ac.uk/) and the final number is expected to remain under 1000, it was reasoned that multiplexing samples would significantly minimize the per sample cost of next generation DNA sequencing and improve experimental design. Four barcoded 3′ adapter oligonucleotides (BC1 to BC4) were designed to be used in multiplex sequencing of miRNAs while retaining sample identity. The barcoded oligonucleotides were pre-adenylated as described herein, using a complementary template that accommodated the degenerate nature of the barcode base pair positions.

The purified, barcoded, 3′ pre-adenylated oligonucleotides were then used to ligate two synthetic miRNA fragments (an 18-mer and a 21-mer) combined at various concentrations in four independent reactions, followed by ligation of the 5′ adapter. The four samples were then combined and the ligation products were reverse transcribed in a single reaction. Following amplification, the resulting pooled fragments were cloned into a vector and sequenced. Analysis of the resulting sequences revealed that this approach could efficiently be used to achieve multiplex analysis of miRNAs from mixed samples (Table 1). Table 1 lists the number of expected and sequenced clones out of 200 randomly selected positive colonies from a single pooled reaction of the four barcoded samples following ligation-based capture of miRNA-18 and miRNA-21 present at various concentration in each samples.

TABLE 1
Multiplex analysis of barcoded miRNAs libraries.

| | |
|---|---|
| miRNA-18 | miRNA-21 |

| | Expected | Sequenced | Expected | Sequenced |
|---|---|---|---|---|
| BC1 | 25 | 24 | 25 | 29 |
| BC2 | 15 | 16 | 35 | 31 |
| BC3 | 35 | 33 | 15 | 12 |
| BC4 | 45 | 52 | 5 | 3 |

To further validate the use of these barcoded adapters in a biological context, a similar ligation-based capture of miRNAs was conducted using human brain total RNA as starting material. Analysis of the resulting sequences revealed that a large proportion of miRNAs were efficiently captured by this approach, while maintaining relative distribution of the barcoded adapters throughout the samples (Table 2). Table 2 lists the distribution of sequenced clones out of 200 randomly selected positive colonies from a single pooled reaction of the four barcoded samples following ligation-based capture of miRNAs extracted from a human brain total RNA sample. The identity of the miRNAs sequenced was validated using the mirBase 11.0 database (Worldwide Website: microrna.sanger.ac.uk/). Actual miRNA-library sequences are listed in Table 3.

TABLE 2
Multiplex analysis of barcoded miRNA libraries
from a human biological sample.

| | Number | | | | |
|---|---|---|---|---|---|
| | of | | | | |
| ID type | clones | | Barcode | | Number of clones |
| miRNA | 88 | 44% | BC1 (ATAT) | 46 | 23% |
| rRNA | 61 | 31% | BC2 (GCGC) | 52 | 26% |
| mRNA/contig | 32 | 16% | BC3 (TAGC) | 54 | 27% |
| snRNA | 19 | 9% | BC4 (CCAA) | 48 | 24% |

TABLE 3

Sequences of barcoded miRNA libraries from a human
biological sample.

| | | |
|---|---|---|
| ID | 5′ adapter-miRNA Barcode-3′ adapter | Barcode |

| | | |
|---|---|---|
| Hsa-let- | AATGATACGGCGACCACCGACAGGTTCAGAGTTCTACAG | BC4 |
| 7a-1 | TCCGACGATCTGAGGTAGTAGGTTGTATAGTCCAATCGT | |
| | ATGCCGTCTTCTGCTTG (SEQ ID NO: 1) | |
| Hsa-let-7b | AATGATACGGCGACCACCGACAGGTTCAGAGTTCTACAG | BC2 |
| | TCCGACGATCTGAGGTAGTAGGTTGTGTGGTTGCGCTCG | |
| | TATGCCGTCTTCTGCTTG (SEQ ID NO: 2) | |
| Hsa-let-7b | AATGATACGGCGACCACCGACAGGTTCAGAGTTCTACAG | BC3 |
| | TCCGACGATCTGAGGTAGTAGGTTGTGTGGTTTAGCTCG | |
| | TATGCCGTCTTCTGCTTG (SEQ ID NO: 3) | |
| Hsa-let-7d | AATGATACGGCGACCACCGACAGGTTCAGAGTTCTACAG | BC1 |
| | TCCGACGATCAAGGAAGGCAGCAGGCGCGCAAATATTC | |
| | GTATGCCGTCTTCTGCTTG (SEQ ID NO: 4) | |
| Hsa-let-7e | AATGATACGGCGACCACCGACAGGTTCAGAGTTCTACAG | BC2 |
| | TCCGACGATCTGAGGTAGGAGGTTGTATAGTTGCGCTCG | |
| | TATGCCGTCTTCTGCTTG (SEQ ID NO: 5) | |
| Hsa-let-7f-1 | AATGATACGGCGACCACCGACAGGTTCAGAGTTCTACAG | BC2 |
| | TCCGACGATCTGAGGTAGTAGATTGTATAGTTGCGCTCG | |
| | TATGCCGTCTTCTGCTTG (SEQ ID NO: 6) | |
| Hsa-miR- | AATGATACGGCGACCACCGACAGGTTCAGAGTTCTACAG | BC2 |
| 100 | TCCGACGATCAACCCGTAGATCCGAACTTGTGGCGCTC | |
| | GTATGCCGTCTTCTGCTTG (SEQ ID NO: 7) | |
| Hsa-miR- | AATGATACGGCGACCACCGACAGGTTCAGAGTTCTACAG | BC1 |
| 101 | TCCGACGATCTACAGTACTGTGATAACTGAAATATTCGT | |
| | ATGCCGTCTTCTGCTTG (SEQ ID NO: 8) | |
| Hsa-miR- | AATGATACGGCGACCACCGACAGGTTCAGAGTTCTACAG | BC3 |
| 103 | TCCGACGATCAGCAGCATTGTACAGGGCTATGATAGCTC | |
| | GTATGCCGTCTTCTGCTTG (SEQ ID NO: 9) | |
| Hsa-miR- | AATGATACGGCGACCACCGACAGGTTCAGAGTTCTACAG | BC3 |
| 106b | TCCGACGATCTAAAGTGCTGACAGTGCAGATTAGCTCGT | |
| | ATGCCGTCTTCTGCTTG (SEQ ID NO: 10) | |
| Hsa-mir- | AATGATACGGCGACCACCGACAGGTTCAGAGTTCTACAG | BC1 |
| 124-1 | TCCGACGATCTAAGGCACGCGGTGAATGCCATATTCGTA | |
| | TGCCGTCTTCTGCTTG (SEQ ID NO: 11) | |
| Hsa-mir- | AATGATACGGCGACCACCGACAGGTTCAGAGTTCTACAG | BC2 |
| 125a | TCCGACGATCTCCCTGAGACCCTTTAACCTGTGCGCTCG | |
| | TATGCCGTCTTCTGCTTG (SEQ ID NO: 12) | |
| Hsa-mir- | AATGATACGGCGACCACCGACAGGTTCAGAGTTCTACAG | BC4 |
| 125b-1 | TCCGACGATCTCCCTGAGACCCTAACTTGTGACCAATCG | |
| | TATGCCGTCTTCTGCTTG (SEQ ID NO: 13) | |
| Hsa-miR- | AATGATACGGCGACCACCGACAGGTTCAGAGTTCTACAG | BC3 |
| 127 | TCCGACGATCCTGAAGCTCAGAGGGCTCTGATTAGCTC | |
| | GTATGCCGTCTTCTGCTTG (SEQ ID NO: 14) | |
| Hsa-miR- | AATGATACGGCGACCACCGACAGGTTCAGAGTTCTACAG | BC3 |
| 127 | TCCGACGATCCTGAAGCTCAGAGGGCTCTGATTAGCTC | |
| | GTATGCCGTCTTCTGCTTG (SEQ ID NO: 15) | |
| Hsa-miR- | AATGATACGGCGACCACCGACAGGTTCAGAGTTCTACAG | BC2 |
| 128a | TCCGACGATCTCCCACCGCTGCCACCCGCGCTCGTATG | |
| | CCGTCTTCTGCTTG (SEQ ID NO: 16) | |
| Hsa-miR- | AATGATACGGCGACCACCGACAGGTTCAGAGTTCTACAG | BC1 |
| 129 | TCCGACGATCAAGCCCTTACCCCAAAAAGTATATATTCG | |
| | TATGCCGTCTTCTGCTTG (SEQ ID NO: 17) | |
| Hsa-miR- | AATGATACGGCGACCACCGACAGGTTCAGAGTTCTACAG | BC3 |
| 130a | TCCGACGATCCAGTGCAATGTTAAAAGGGCATTAGCTCG | |
| | TATGCCGTCTTCTGCTTG (SEQ ID NO: 18) | |
| Hsa-mir- | AATGATACGGCGACCACCGACAGGTTCAGAGTTCTACAG | BC4 |
| 132 | TCCGACGATCTAACAGTCTACAGCCATGGTCGCCAATCG | |
| | TATGCCGTCTTCTGCTTG (SEQ ID NO: 19) | |
| Hsa-miR- | AATGATACGGCGACCACCGACAGGTTCAGAGTTCTACAG | BC4 |
| 142-5p | TCCGACGATCCATAAAGTAGAAAGCACTACTCCAATCGT | |
| | ATGCCGTCTTCTGCTTG (SEQ ID NO: 20) | |
| Hsa-mir- | AATGATACGGCGACCACCGACAGGTTCAGAGTTCTACAG | BC1 |
| 143 | TCCGACGATCTGAGATGAAGCACTGTAGCTCTATATTCG | |
| | TATGCCGTCTTCTGCTTG (SEQ ID NO: 21) | |
| Hsa-miR- | AATGATACGGCGACCACCGACAGGTTCAGAGTTCTACAG | BC1 |
| 146b | TCCGACGATCTGCCCTGTGGACTCAGTTCTGGATATTCG | |
| | TATGCCGTCTTCTGCTTG (SEQ ID NO: 22) | |
| Hsa-miR- | AATGATACGGCGACCACCGACAGGTTCAGAGTTCTACAG | BC1 |
| 149 | TCCGACGATCTCTGGCTCCGTGTCTTCACTCCCATATTC | |
| | GTATGCCGTCTTCTGCTTG (SEQ ID NO: 23) | |
| Hsa-miR- | AATGATACGGCGACCACCGACAGGTTCAGAGTTCTACAG | BC3 |
| 150 | TCCGACGATCTCTCCCAACCCTTGTACCAGTGTAGCTCG | |
| | TATGCCGTCTTCTGCTTG (SEQ ID NO: 24) | |
| Hsa-miR- | AATGATACGGCGACCACCGACAGGTTCAGAGTTCTACAG | BC4 |
| 15a | TCCGACGATCTAGCAGCACATAATGGTTTGTGCCAATCG | |
| | TATGCCGTCTTCTGCTTG (SEQ ID NO: 25) | |
| Hsa-miR- | AATGATACGGCGACCACCGACAGGTTCAGAGTTCTACAG | BC2 |
| 16 | TCCGACGATCTAGCAGCACGTAAATATTGGCGGCGCTC | |
| | GTATGCCGTCTTCTGCTTG (SEQ ID NO: 26) | |
| Hsa-mir- | AATGATACGGCGACCACCGACAGGTTCAGAGTTCTACAG | BC3 |
| 181a-2 | TCCGACGATCAACATTCAACGCTGTCGGTGAGTTAGCTC | |
| | GTATGCCGTCTTCTGCTTG (SEQ ID NO: 27) | |
| Hsa-mir- | AATGATACGGCGACCACCGACAGGTTCAGAGTTCTACAG | BC4 |
| 181c | TCCGACGATCAACATTCAACGCTGTCGGTGACCAATCGT | |
| | ATGCCGTCTTCTGCTTG (SEQ ID NO: 28) | |
| Hsa-mir- | AATGATACGGCGACCACCGACAGGTTCAGAGTTCTACAG | BC2 |
| 1826 | TCCGACGATCATTGATCATCGACACTTCGAACGCACTTG | |
| | CGGCCCCGGGTTGCGCTCGTATGCCGTCTTCTGCTTG | |
| | (SEQ ID NO: 29) | |
| Hsa-mir- | AATGATACGGCGACCACCGACAGGTTCAGAGTTCTACAG | BC3 |
| 1826 | TCCGACGATCCATTGATCATCGACACTTCGAACGCACTT | |
| | GCGGCCCCGGGTTTAGCTCGTATGCCGTCTTCTGCTTG | |
| | (SEQ ID NO: 30) | |
| Hsa-miR- | AATGATACGGCGACCACCGACAGGTTCAGAGTTCTACAG | BC2 |
| 185 | TCCGACGATCTGGAGAGAAAGGCAGTTCCTGAGCGCTC | |
| | GTATGCCGTCTTCTGCTTG (SEQ ID NO: 31) | |
| Hsa-miR- | AATGATACGGCGACCACCGACAGGTTCAGAGTTCTACAG | BC1 |
| 186 | TCCGACGATCCAAAGAATTCTCCTTTTGGGCTATATTCGT | |
| | ATGCCGTCTTCTGCTTG (SEQ ID NO: 32) | |
| Hsa-miR- | AATGATACGGCGACCACCGACAGGTTCAGAGTTCTACAG | BC1 |
| 187 | TCCGACGATCTCGTGTCTTGTGTTGCAGCCGGATATTCG | |
| | TATGCCGTCTTCTGCTTG (SEQ ID NO: 33) | |
| Hsa-miR- | AATGATACGGCGACCACCGACAGGTTCAGAGTTCTACAG | BC1 |
| 188 | TCCGACGATCCTCCCACATGCAGGGTTTGCAATATTCGT | |
| | ATGCCGTCTTCTGCTTG (SEQ ID NO: 34) | |
| Hsa-miR- | AATGATACGGCGACCACCGACAGGTTCAGAGTTCTACAG | BC4 |
| 188 | TCCGACGATCCTCCCACATGCAGGGTTTGCACCAATCGT | |
| | ATGCCGTCTTCTGCTTG (SEQ ID NO: 35) | |
| Hsa-miR- | AATGATACGGCGACCACCGACAGGTTCAGAGTTCTACAG | BC4 |
| 191 | TCCGACGATCCAACGGAATCCCAAAAGCAGCTGCCAAT | |
| | CGTATGCCGTCTTCTGCTTG (SEQ ID NO: 36) | |
| Hsa-miR- | AATGATACGGCGACCACCGACAGGTTCAGAGTTCTACAG | BC1 |
| 196a | TCCGACGATCTAGGTAGTTTCATGTTGTTGGGATATTCGT | |
| | ATGCCGTCTTCTGCTTG (SEQ ID NO: 37) | |
| Hsa-miR- | AATGATACGGCGACCACCGACAGGTTCAGAGTTCTACAG | BC3 |
| 202 | TCCGACGATCAGAGGTATAGGGCATGGGAATAGCTCGT | |
| | ATGCCGTCTTCTGCTTG (SEQ ID NO: 38) | |
| Hsa-miR- | AATGATACGGCGACCACCGACAGGTTCAGAGTTCTACAG | BC3 |
| 202 | TCCGACGATCAGAGGTATAGGGCATGGGAATAGCTCGT | |
| | ATGCCGTCTTCTGCTTG (SEQ ID NO: 39) | |
| Hsa-miR- | AATGATACGGCGACCACCGACAGGTTCAGAGTTCTACAG | BC2 |
| 205 | TCCGACGATCTCCTTCATTCCACCGGAGTCTGGCGCTCG | |
| | TATGCCGTCTTCTGCTTG (SEQ ID NO: 40) | |
| Hsa-miR- | AATGATACGGCGACCACCGACAGGTTCAGAGTTCTACAG | BC3 |
| 20a | TCCGACGATCTAAAGTGCTTATAGTGCAGGTAGTAGCTC | |
| | GTATGCCGTCTTCTGCTTG (SEQ ID NO: 41) | |
| Hsa-miR- | AATGATACGGCGACCACCGACAGGTTCAGAGTTCTACAG | BC2 |
| 20b | TCCGACGATCCAAAGTGCTCATAGTGCAGGTAGGCGCT | |
| | CGTATGCCGTCTTCTGCTTG (SEQ ID NO: 42) | |
| Hsa-miR- | AATGATACGGCGACCACCGACAGGTTCAGAGTTCTACAG | BC1 |
| 212 | TCCGACGATCTAACAGTCTCCAGTCACGGCCATATTCGT | |
| | ATGCCGTCTTCTGCTTG (SEQ ID NO: 43) | |
| Hsa-mir- | AATGATACGGCGACCACCGACAGGTTCAGAGTTCTACAG | BC3 |
| 216b | TCCGACGATCTCAGAGTTCTACAGTCTGATAGCTCGTAT | |
| | GCCGTCTTCTGCTTG (SEQ ID NO: 44) | |
| Hsa-mir- | AATGATACGGCGACCACCGACAGGTTCAGAGTTCTACAG | BC4 |
| 218-1 | TCCGACGATCTTGTGCTTGATCTAACCATGTGACCAATC | |
| | GTATGCCGTCTTCTGCTTG (SEQ ID NO: 45) | |
| Hsa-mir- | AATGATACGGCGACCACCGACAGGTTCAGAGTTCTACAG | BC1 |
| 219-2 | TCCGACGATCAGAATTGTGGCTGGACATCTGTATATTCG | |
| | TATGCCGTCTTCTGCTTG (SEQ ID NO: 46) | |
| Hsa-miR- | AATGATACGGCGACCACCGACAGGTTCAGAGTTCTACAG | BC3 |
| 22 | TCCGACGATCAAGCTGCCAGTTGAAGAACTGTTAGCTCG | |
| | TATGCCGTCTTCTGCTTG (SEQ ID NO: 47) | |
| Hsa-miR- | AATGATACGGCGACCACCGACAGGTTCAGAGTTCTACAG | BC3 |
| 221 | TCCGACGATCAGCTACATTGTCTGCTGGGTTTCTAGCTC | |
| | GTATGCCGTCTTCTGCTTG (SEQ ID NO: 48) | |
| Hsa-miR- | AATGATACGGCGACCACCGACAGGTTCAGAGTTCTACAG | BC1 |
| 222 | TCCGACGATCAGCTACATCTGGCTACTGGGTATATTCGT | |
| | ATGCCGTCTTCTGCTTG (SEQ ID NO: 49) | |
| Hsa-miR- | AATGATACGGCGACCACCGACAGGTTCAGAGTTCTACAG | BC1 |
| 23a | TCCGACGATCATCACATTGCCAGGGATTTCCATATTCGT | |
| | ATGCCGTCTTCTGCTTG (SEQ ID NO: 50) | |
| Hsa-mir- | AATGATACGGCGACCACCGACAGGTTCAGAGTTCTACAG | BC3 |
| 25 | TCCGACGATCCATTGCACTTGTCTCGGTCTGATAGCTCG | |
| | TATGCCGTCTTCTGCTTG (SEQ ID NO: 51) | |
| Hsa-mir- | AATGATACGGCGACCACCGACAGGTTCAGAGTTCTACAG | BC2 |
| 26a-1 | TCCGACGATCTTCAAGTAATCCAGGATAGGCAGCGCTC | |
| | GTATGCCGTCTTCTGCTTG (SEQ ID NO: 52) | |
| Hsa-mir- | AATGATACGGCGACCACCGACAGGTTCAGAGTTCTACAG | BC4 |
| 26a-1 | TCCGACGATCCAAGTAATCCAGGATAGGCTTCCAATCGT | |
| | ATGCCGTCTTCTGCTTG (SEQ ID NO: 53) | |
| Hsa-miR- | AATGATACGGCGACCACCGACAGGTTCAGAGTTCTACAG | BC2 |
| 26b | TCCGACGATCTTCAAGTAATTCAGGATAGGTGCGCTCGT | |
| | ATGCCGTCTTCTGCTTG (SEQ ID NO: 54) | |
| Hsa-miR- | AATGATACGGCGACCACCGACAGGTTCAGAGTTCTACAG | BC4 |
| 27b | TCCGACGATCTTCACAGTGGCTAAGTTCTGCCCAATCGT | |
| | ATGCCGTCTTCTGCTTG (SEQ ID NO: 55) | |
| Hsa-miR- | AATGATACGGCGACCACCGACAGGTTCAGAGTTCTACAG | BC2 |
| 302d | TCCGACGATCTAAGTGCTTCCATGTTTGAGTGTGCGCTC | |
| | GTATGCCGTCTTCTGCTTG (SEQ ID NO: 56) | |
| Hsa-miR- | AATGATACGGCGACCACCGACAGGTTCAGAGTTCTACAG | BC2 |
| 30a-5p | TCCGACGATCCTTTCAGTCGGATGTTTGCAGCGCGCTCG | |
| | TATGCCGTCTTCTGCTTG (SEQ ID NO: 57) | |
| Hsa-miR- | AATGATACGGCGACCACCGACAGGTTCAGAGTTCTACAG | BC4 |
| 30b | TCCGACGATCTGTAAACATCCTACACTCAGCTCCAATCG | |
| | TATGCCGTCTTCTGCTTG (SEQ ID NO: 58) | |
| Hsa-miR- | AATGATACGGCGACCACCGACAGGTTCAGAGTTCTACAG | BC2 |
| 328 | TCCGACGATCCTGGCCCTCTCTGCCCTTCCGTGCGCTCG | |
| | TATGCCGTCTTCTGCTTG (SEQ ID NO: 59) | |
| Hsa-miR- | AATGATACGGCGACCACCGACAGGTTCAGAGTTCTACAG | BC3 |
| 337 | TCCGACGATCCTCCTATATGATGCCTTTCTTCTAGCTCGT | |
| | ATGCCGTCTTCTGCTTG (SEQ ID NO: 60) | |
| Hsa-miR- | AATGATACGGCGACCACCGACAGGTTCAGAGTTCTACAG | BC3 |
| 340 | TCCGACGATCTTATAAAGCAATGAGACTGATTTAGCTCG | |
| | TATGCCGTCTTCTGCTTG (SEQ ID NO: 61) | |
| Hsa-miR- | AATGATACGGCGACCACCGACAGGTTCAGAGTTCTACAG | BC1 |
| 367 | TCCGACGATCAATTGCACTTTAGCAATGGTGAATATTCG | |
| | TATGCCGTCTTCTGCTTG (SEQ ID NO: 62) | |
| Hsa-miR- | AATGATACGGCGACCACCGACAGGTTCAGAGTTCTACAG | BC2 |
| 369-3p | TCCGACGATCAATAATACATGGTTGATCTTTGCGCTCGT | |
| | ATGCCGTCTTCTGCTTG (SEQ ID NO: 63) | |
| Hsa-miR- | AATGATACGGCGACCACCGACAGGTTCAGAGTTCTACAG | BC2 |
| 369-5p | TCCGACGATCAGATCGACCGTGTTATATTCGCGCGCTCG | |
| | TATGCCGTCTTCTGCTTG (SEQ ID NO: 64) | |
| Hsa-miR- | AATGATACGGCGACCACCGACAGGTTCAGAGTTCTACAG | BC4 |
| 370 | TCCGACGATCGCCTGCTGGGGTGGAACCTGGTCCAATC | |
| | GTATGCCGTCTTCTGCTTG (SEQ ID NO: 65) | |
| Hsa-miR- | AATGATACGGCGACCACCGACAGGTTCAGAGTTCTACAG | BC3 |
| 376b | TCCGACGATCATCATAGAGGAAAATCCATGTTTAGCTCG | |
| | TATGCCGTCTTCTGCTTG (SEQ ID NO: 66) | |
| Hsa-mir- | AATGATACGGCGACCACCGACAGGTTCAGAGTTCTACAG | BC3 |
| 423 | TCCGACGATCAAGCTCGGTCTGAGGCCCCTCAGTTAGCT | |
| | CGTATGCCGTCTTCTGCTTG (SEQ ID NO: 67) | |
| Hsa-miR- | AATGATACGGCGACCACCGACAGGTTCAGAGTTCTACAG | BC4 |
| 424 | TCCGACGATCCAGCAGCAATTCATGTTTTGAACCAATCG | |
| | TATGCCGTCTTCTGCTTG (SEQ ID NO: 68) | |
| Hsa-miR- | AATGATACGGCGACCACCGACAGGTTCAGAGTTCTACAG | BC2 |
| 450 | TCCGACGATCTTTTGCGATGTGTTCCTAATATGCGCTCGT | |
| | ATGCCGTCTTCTGCTTG (SEQ ID NO: 69) | |
| Hsa-miR- | AATGATACGGCGACCACCGACAGGTTCAGAGTTCTACAG | BC1 |
| 452 | TCCGACGATCAACTGTTTGCAGAGGAAACTGAATATTCG | |
| | TATGCCGTCTTCTGCTTG (SEQ ID NO: 70) | |
| Hsa-miR- | AATGATACGGCGACCACCGACAGGTTCAGAGTTCTACAG | BC2 |
| 483 | TCCGACGATCTCACTCCTCTCCTCCCGTCTTGCGCTCGT | |
| | ATGCCGTCTTCTGCTTG (SEQ ID NO: 71) | |
| Hsa-miR- | AATGATACGGCGACCACCGACAGGTTCAGAGTTCTACAG | BC3 |
| 486 | TCCGACGATCCGGGGCAGCTCAGTACAGGATTAGCTCG | |
| | TATGCCGTCTTCTGCTTG (SEQ ID NO: 72) | |
| Hsa-miR- | AATGATACGGCGACCACCGACAGGTTCAGAGTTCTACAG | BC3 |
| 487b | TCCGACGATCAATCGTACAGGGTCATCCACTTTAGCTCG | |
| | TATGCCGTCTTCTGCTTG (SEQ ID NO: 73) | |
| Hsa-miR- | AATGATACGGCGACCACCGACAGGTTCAGAGTTCTACAG | BC4 |
| 495 | TCCGACGATCAAACAAACATGGTGCACTTCTTCCAATCG | |
| | TATGCCGTCTTCTGCTTG (SEQ ID NO: 74) | |
| Hsa-miR- | AATGATACGGCGACCACCGACAGGTTCAGAGTTCTACAG | BC4 |
| 497 | TCCGACGATCCAGCAGCACACTGTGGTTTGTCCAATCGT | |
| | ATGCCGTCTTCTGCTTG (SEQ ID NO: 75) | |
| Hsa-miR- | AATGATACGGCGACCACCGACAGGTTCAGAGTTCTACAG | BC3 |
| 501 | TCCGACGATCAATGCACCCGGGCAAGGATTCTTAGCTC | |
| | GTATGCCGTCTTCTGCTTG (SEQ ID NO: 76) | |
| Hsa-miR- | AATGATACGGCGACCACCGACAGGTTCAGAGTTCTACAG | BC4 |
| 501 | TCCGACGATCAATGCACCCGGGCAAGGATTCTCCAATC | |
| | GTATGCCGTCTTCTGCTTG (SEQ ID NO: 77) | |
| Hsa-miR- | AATGATACGGCGACCACCGACAGGTTCAGAGTTCTACAG | BC1 |
| 503 | TCCGACGATCTAGCAGCGGGAACAGTTCTGCAGATATTC | |
| | GTATGCCGTCTTCTGCTTG (SEQ ID NO: 78) | |
| Hsa-miR- | AATGATACGGCGACCACCGACAGGTTCAGAGTTCTACAG | BC3 |
| 503 | TCCGACGATCTAGCAGCGGGAACAGTTCTGCAGTAGCT | |
| | CGTATGCCGTCTTCTGCTTG (SEQ ID NO: 79) | |
| Hsa-miR- | AATGATACGGCGACCACCGACAGGTTCAGAGTTCTACAG | BC2 |
| 539 | TCCGACGATCGGAGAAATTATCCTTGGTGTGTGCGCTCG | |
| | TATGCCGTCTTCTGCTTG (SEQ ID NO: 80) | |
| Hsa-mir- | AATGATACGGCGACCACCGACAGGTTCAGAGTTCTACAG | BC3 |
| 598 | TCCGACGATCTACGTCATCGTTGTCATCGTCATAGCTCG | |
| | TATGCCGTCTTCTGCTTG (SEQ ID NO: 106) | |
| Hsa-miR-7 | AATGATACGGCGACCACCGACAGGTTCAGAGTTCTACAG | BC3 |
| | TCCGACGATCTGGAAGACTAGTGATTTTGTTGTTAGCTC | |
| | GTATGCCGTCTTCTGCTTG (SEQ ID NO: 81) | |
| Hsa-mir- | AATGATACGGCGACCACCGACAGGTTCAGAGTTCTACAG | BC1 |
| 889 | TCCGACGATCTTAATATCGGACAACCATTGTATATTCGTA | |
| | TGCCGTCTTCTGCTTG (SEQ ID NO: 82) | |
| Hsa-miR- | AATGATACGGCGACCACCGACAGGTTCAGAGTTCTACAG | BC3 |
| 92 | TCCGACGATCTATTGCACTTGTCCCGGCCTGTTAGCTCG | |
| | TATGCCGTCTTCTGCTTG (SEQ ID NO: 83) | |
| Hsa-miR- | AATGATACGGCGACCACCGACAGGTTCAGAGTTCTACAG | BC2 |
| 93 | TCCGACGATCCAAAGTGCTGTTCGTGCAGGTAGGCGCT | |
| | CGTATGCCGTCTTCTGCTTG (SEQ ID NO: 84) | |
| Hsa-miR- | AATGATACGGCGACCACCGACAGGTTCAGAGTTCTACAG | BC2 |
| 98 | TCCGACGATCTGAGGTAGTAAGTTGTATTGTTGCGCTCG | |
| | TATGCCGTCTTCTGCTTG (SEQ ID NO: 85) | |
| Hsa-miR- | AATGATACGGCGACCACCGACAGGTTCAGAGTTCTACAG | BC3 |
| 98 | TCCGACGATCTGAGGTAGTAAGTTGTATTGTTTAGCTCG | |
| | TATGCCGTCTTCTGCTTG (SEQ ID NO: 86) | |
| Hsa-miR- | AATGATACGGCGACCACCGACAGGTTCAGAGTTCTACAG | BC4 |
| 99a | TCCGACGATCAACCCGTAGATCCGATCTTGTGCCAATCG | |
| | TATGCCGTCTTCTGCTTG (SEQ ID NO: 87) | |

### Example III

### Discussion

A facile approach to produce pre-adenylated, barcoded oligonucleotides suitable for efficient miRNA capture, as well as methods for sequencing by ligation and multiplex analyses are described herein. The yield of pre-adenylation achieved by this approach and the simplicity of this method is a significant improvement compared to the chemical synthesis process conventionally used in the art. MiRNA and RNA end capture experiments will greatly benefit from the speed, convenience, and accessibility of methods and compositions described herein. Further, the methods and compositions described herein provide one of skill in the art the ability to use adapters of any sequence. Supposing a 100-fold variation between the abundance of low and high miRNAs expression, one of skill in the art could easily combine between 50 to 150 different samples, depending on the cyclic array technology used, in a quantitative expression profiling of miRNAs. This would result in a significant cost reduction associated with the use of next-generation sequencing and will facilitate studies involving multiple conditions and/or time course experiments. Moreover, the optimized ligation conditions and the use of barcoded adapters described herein favors the design of more complex experiments and the achievement of higher yield of miRNA capture, which will likely result in the identification of undiscovered miRNAs and a better understanding of their implication in cellular processes.

### Example IV

### Materials and Methods

Initial Pre-Adenylation of the 3′ Adapter Oligonucleotide with T4 DNA Ligase 1

34-mer oligonucleotide pD was annealed to its complementary 40-mer template by incubating 10 μl of 100 μM of each oligonucleotides at 90° C. for 3 minutes and allowing the mixture to cool to room temperature over a 60 minute period. For initial pre-adenylation and time course experiments, 10 pmoles of the annealed oligonucleotide was incubated with 5 μl of 2× Quick Ligation Reaction Buffer and 1 μl of T4 DNA ligase (2000 U/μl, NEB) in a final volume of 10 μl at 37° C. for the indicated time (FIG. 4). The reaction was stopped by heat inactivation at 65° C. for 15 minutes. All denaturing polyacrylamide TBE-urea gel experiments were conducted as follows: Novex TBE-Urea Sample Buffer (2×) (Invitrogen) was added to the samples, followed by 3 minute incubation at 90° C. and put on ice prior to loading. 10 μl were then loaded on a pre-cast 10% or 15% Novex® TBE-Urea Gels (Invitrogen), and were ran at 15 watts for 12 to 15 minutes in pre-warmed running buffer. The gels were stained in a SYBR Gold Nucleic Acid Gel Stain (5 μl in 150 ml of TBE, Invitrogen) for 15 minutes and visualized on a Gel Doc 2000 (Bio-Rad).

Scale-Up Pre-Adenylation of the 3′ Adapter Oligonucleotide with T4 DNA Ligase 1

In order to produce sufficient pre-adenylated oligonucleotide for experiments described herein, 500 pmoles of the annealed oligonucleotide was incubated with 25 μl of 2× Quick Ligation Reaction Buffer (NEB), 1 μl of 10 mM ATP and 5 μl of T4 DNA ligase (2000 U/μl, NEB) in a final volume of 50 μl at 37° C. for 60 minutes. 5 μl of T4 DNA ligase (2000 U/μl, NEB), and 1 μl of 10 mM ATP were added a second time and returned to 37° C. for another 60 minutes. The reaction mixture was mixed every 20 minutes throughout the 120 minute incubation time. The reaction was stopped by heat inactivation at 65° C. for 15 minutes.

### Gel Purification

The pre-adenylation reaction mixture was loaded on four 15% 2D well Novex® TBE-Urea Gels (Invitrogen) as described above. The band corresponding to the pre-adenylated oligonucleotide (AppD) was then excised and extracted from the gel. The gel slices were pulverized together by centrifugation through a needle hole at the bottom of a 0.5 mL tube placed in a 1.5 mL tube. 800 μl of dH₂O was added to the pulverized gel slices, vortexed and incubated at 70° C. for 30 minutes, while vortexing the sample every 10 minutes. The gel slurry was transferred to a 0.2 μm Nanosep tube (Pall Corporation) and filtered by centrifugation. The mixture was sec-butanol extracted to approximately 300 to 400 μl followed by extraction with one volume of phenol:chloroform:isoamyl alcohol (25:24:1, v/v), one volume of chloroform, and precipitated with 2 μL of 20 mg/ml glycogen, 1/10 volume of 3M NaOAc (pH 5.2) and 2.5× of 100% cold ethanol. Samples were frozen for 20 minutes on dry ice, and then centrifuged for 20 minutes at maximum speed. Following ethanol precipitation, samples were resuspended in an appropriate volume of dH₂O as required.

### Alternative Purification Methods

In order to purify large quantities of pre-adenylated oligonucleotides, an alternative to gel purification was developed. The complementary oligonucleotide “template” was purchased with a biotin group on its 5′ termini. 125 μl (approximately 1 mg) of Dynabeads® MyOne™ Streptavidin C1 (Invitrogen) was pre-washed (three washes with 250 μl of 1× bind and wash buffer) and resuspended in 250 μl of 2× bind and wash buffer according to manufacturer specifications. Following annealing and pre-adenylation of the oligonucleotide as explained above, 200 μl of dH₂O was added to the reaction mixture followed by approximately 1 mg of washed Dynabeads® MyOne™ Streptavidin C1. The paramagnetic beads were incubated for 15 minutes at room temperature under gentle agitation and then placed on a magnet for 2 minutes to pellet the beads. The beads were washed three times with 250 μl of 1× bind and wash buffer to remove any residual enzyme, ATP and unbound oligonucleotides. The beads were resuspended with 125 μl of ice cold 100 mM NaOH and incubated on ice for 3 minutes and subjected to magnetic separation for 1 minute. The supernatant (which contained the pre-adenylated oligonucleotide) was moved to a clean tube without disturbing the beads. 125 μl of 150 mM HCl was quickly added to the supernatant followed by addition of 200 μl of 1×TE. The supernatant was ethanol precipitated, resuspended in 50 μl of dH₂O and the resulting was quantitated using a NanoDrop™ 1000 spectrophotometer (Thermo Fisher Scientific). Finally, 1 pmole was subjected to PAGE to verify successful pre-adenylation and purification. It was routinely observed that a small fraction of the biotinylated oligonucleotide detached from beads during NaOH denaturation. However, this slight contamination did not adversely affect ligation of the 3′ adapter. Nevertheless a second round of binding to fresh beads will capture any residual complementary template.

### Ligation of the Pre-Adenylated 3′ Adapter to the Synthetic MiRNA Using T4 RNA Ligase 2 (RNL2)

Unless stated otherwise, ligation reactions used 10 pmoles of synthetic miRNA (pA), 10 pmoles of pre-adenylated 3′ adapter (AppD), 2 μl of 10×T4 RNL2 truncated reaction buffer (which lacks ATP, NEB), 2.4 μl of polyethylene glycol 8000 (Sigma) and 1 μl of T4 RNA ligase 2 (RNL2) (200 U/μl, NEB) in a final volume of 20 μl at 37° C. for 60 minutes. The reactions were quenched with loading buffer.

Ligation of the 5′ Adapters to the miRNA-3′ Adapter Product Using T4 RNA Ligase 1

Ligation of the 5′ adapters was conducted using the miRNA-3′ adapter either PAGE purified or phenol chloroform extracted and ethanol precipitated. In either case, the ligated product was incubated with 100 pmoles of 5′ adapter, 2 μl of 10×T4 RNA ligase 1 reaction buffer (which contains ATP, NEB), 3 μl of 100% DMSO (Sigma) and 1 μl of T4 RNA ligase 1 (20 U/μl, NEB) in a final volume of 20 μl at 37° C. for 60 minutes (it was critical to denature the reaction mixture at 90° C. for 30 seconds and immediately cool down on ice prior adding the T4 RNA ligase). The reactions were quenched with loading buffer.

### Pre-Adenylation of Barcoded Oligonucleotides

25-mer 3′ adapters were designed with a four nucleotide barcode at their 3′ termini (BC1 to BC4). These oligonucleotides were annealed in independent reactions with a 36-mer complementary template having four degenerate nucleotides positioned for pairing with the barcodes on each oligonucleotide. The four oligonucleotides were pre-adenylated, purified, and used in four independent 3′ ligation experiments in which two synthetic miRNA oligonucleotides were mixed at various concentrations (miRNA-18: miRNA-21; BC1 5:5 pmoles, BC2 3:7 pmoles, BC3 7:3 pmoles, BC4 9:1 pmoles). Following direct 5′ adapter ligation without prior PAGE purification, the ligation products were then pooled together in one single reaction, reverse transcribed, and amplified as described (Pak and Fire (2007) Science 315(5809):241). The resulting amplified products were cloned into Zero Blunt® TOPO® PCR Cloning Kit for Sequencing with One Shot® TOP10 chemically competent E. coli (Invitrogen), as detailed by the manufacturer. Colonies were randomly picked, purified and sequenced (Genomic Solutions, Agencourt) to achieve 200 sequences positive for inserts. The proportion of each barcoded library sequenced in relation to the expected initial pooled concentration for each miRNA oligonucleotides is indicated in Table 1.

### Note on Pre-Adenylation of the Barcoded Oligonucleotides

If using a degenerate complementary template to anneal to the barcoded oligonucleotides, it is preferable to use a large excess of template instead of a 1:1 ratio, since most of the degenerate sequences will not anneal efficiently and will result in a reaction mixture of incomplete adenylation. While certain experiments described herein were performed at a 1:1 ratio resulting in a pool of pre-adenylated and non-adenylated oligonucleotides, efficient 3′ adapter ligation was still observed. When using just a few barcoded oligonucleotides the use of perfectly matched complementary template should be used instead, to ensure a clean purification of near perfect pre-adenylated oligonucleotide.

Multiplex Analysis of Barcoded MiRNA Libraries from a Human Biological Sample

The four pre-adenylated barcode 3′ adapter oligonucleotides produced earlier were further validated in their capacity to ligate miRNAs of a biological sample. 20 μg of total human brain RNA (FirstChoice® Human Brain Reference RNA, Ambion) was PAGE purified using a flashPAGE™ Fractionator (Ambion) to extract all RNAs under approximately 40-mer long. The fraction was then equally divided into four reactions to be ethanol precipitated overnight as recommended by the manufacturer and resuspended in 10 μl of DEPC H₂O. Each reaction was separately subjected to 3′ adapter ligation using one of the pre-adenylated barcode adapters as described herein. Following direct 5′ adapter ligation, the ligation products were then pooled together into one single reaction, PAGE purified, reverse transcribed, and amplified as described by Pak and Fire (Supra). The resulting amplified products were PAGE purified and cloned into Zero Blunt® TOPO® PCR Cloning Kit for Sequencing with One Shot® TOP10 chemically competent E. coli (Invitrogen), as detailed by the manufacturer (PAGE purification following PCR amplification is critical to remove any 5′ adapters directly ligated to 3′ adapters with no miRNA insert). Colonies were randomly picked, purified and sequenced (Genomic Solutions, Agencourt) to achieve 200 sequences positive for inserts. The proportions of each barcode as well as the type of small RNAs sequenced from these pooled libraries of human brain RNA are indicated in Table 2. From these 200 sequences, the 88 sequences demonstrating ligation-based capture of miRNAs are shown in Table 3.

### List of Oligonucleotides

The following oligonucleotides used as described herein were purchased from Integrated DNA Technology. No purification step other than desalting was carried out. The barcoded and complementary degenerated nucleotides are indicated in bold; 5Phos represents a 5′ phosphate; 3 AmM represents a 3′ amino modifier.

(SEQ ID NO: 88)
Synthetic miRNA-21: (Ap)5′ - /5Phos/rCrUrC rArGrG rArUrG rGrCrG rGrArG rCrGrG rUrCrU - 3′.
(SEQ ID NO: 89)
Synthetic miRNA-21: (A) 5′ - rCrUrC rArGrG rArUrG rGrCrG rGrArG rCrGrG rUrCrU - 3′.
(SEQ ID NO: 90)
Synthetic miRNA-18: 5′ - /5Phos/rCrUrC rArGrG rArUrG rGrArG rCrGrG rUrCrU - 3′.
(SEQ ID NO: 91)
pD (3′ adapter): 5′ - /5Phos/AGA TCG GAA GAG CTC GTA TGC CGT CTT CTG CTT G/3AmM/ - 3′.
(SEQ ID NO: 92)
pD-OH: 5′ - /5Phos/AGA TCG GAA GAG CTC GTA TGC CGT CTT CTG CTT G - 3′.
(SEQ ID NO: 93)
pD-PO₄: 5′ - /5Phos/AGA TCG GAA GAG CTC GTA TGC CGT CTT CTG CTT G/3Phos/ - 3′.
(SEQ ID NO: 94)
40-mer Template: 5′ - CAA GCA GAA GAC GGC ATA CGA GCT CTT CCG ATC TTA TAG TGA GTC - 3′.
(SEQ ID NO: 95)
5′ adapter DNA: 5′ - GTT CAG AGT TCT ACA GTC CGA CGA TC - 3′.
(SEQ ID NO: 96)
5′ adapter RNA: 5′ - rGrUrU rCrArG rArGrU rUrCrU rArCrA rGrUrC rCrGrA rCrGrA rUrC - 3′.
(SEQ ID NO: 97)
5′ adapter DNA/RNA: 5′ - GTT CAG AGT TCT ACA rGrUrC rCrGrA rCrGrA rUrC - 3′.
(SEQ ID NO: 98)
3′ adapter Barcode 1: 5′ - /5Phos/ATA TTC GTA TGC CGT CTT CTG CTT G/3AmM/ - 3′.
(SEQ ID NO: 99)
3′ adapter Barcode 2: 5′ - /5Phos/GCG CTC GTA TGC CGT CTT CTG CTT G/3AmM/ - 3′.
(SEQ ID NO: 100)
3′ adapter Barcode 3: 5′ - /5Phos/TAG CTC GTA TGC CGT CTT CTG CTT G/3AmM/ - 3′.
(SEQ ID NO: 101)
3′ adapter Barcode 4 5′ - /5Phos/CCA ATC GTA TGC CGT CTT CTG CTT G/3AmM/ - 3′.
(SEQ ID NO: 102)
Degenerate Template 5′ - CAA GCA GAA GAC GGC ATA CGA NNN NTA TAG TGA GTC - 3′.
(SEQ ID NO: 103)
RT 3′ adapter: 5′ - CAA GCA GAA GAC GGC ATA CGA - 3′.
(SEQ ID NO: 104)
PCR up: 5′ - AAT GAT ACG GCG ACC ACC GAC AGG TTC AGA GTT CTA CAG TCC GA - 3′.
(SEQ ID NO: 105)

PCR low: 5′ - CAA GCA GAA GAC GGC ATA CGA - 3′.

### REFERENCES

1. Lehman (1974) Science 186(4166):790
2. Ohtsuka et al. (1976) Nucl. Acids Res. 3(6):1613
3. McLaughlin et al. (1985) Biochemistry 24(2):267)
4. Patel et al. (2008) Bioorg. Chem. 36(2):46
5. Wang and Silverman (2006) RNA 12(6):1142
6. Chiuman and Li (2002) Bioorg. Chem. 30(5):332
7. Silverman (2004) RNA 10(4):731
8. Wood et al. (2004) Mol. Cell. 13(4):455

It is to be understood that the embodiments of the present invention which have been described are merely illustrative of some of the applications of the principles of the present invention. Numerous modifications may be made by those skilled in the art based upon the teachings presented herein without departing from the true spirit and scope of the invention.

What is claimed is:

1. A method of generating a pre-adenylated oligonucleotide comprising the steps of:
a) providing a first oligonucleotide having a 3′ block and a 5′ phosphate;
b) providing a second oligonucleotide that is partially complementary to the first oligonucleotide;
c) allowing the first oligonucleotide and the second oligonucleotide to hybridize to form a duplex, wherein the second oligonucleotide has a 3′ overhang;
d) contacting the duplex with a DNA ligase and ATP; and
e) allowing the ligase to adenylate the first oligonucleotide to form a pre-adenylated oligonucleotide.

2. The method of claim 1, wherein the DNA ligase is T4 DNA ligase.

3. The method of claim 1, further comprising the step of:
f) purifying the adenylated oligonucleotide.

4. The method of claim 3, wherein the step of purifying is performed by gel electrophoresis.

5. The method of claim 3, wherein the second oligonucleotide has a label and wherein the step of purifying is performed by binding the label.

6. The method of claim 5, wherein the label can bind to a column or a bead.

7. The method of claim 6, wherein the bead is a magnetic bead.

8. A method for retrieving a nucleic acid sequence from a sample comprising the steps of:
a) providing the pre-adenylated oligonucleotide of claim 1;
b) contacting the pre-adenylated oligonucleotide to a sample in the presence of ligase and in the absence of ATP;
c) allowing the pre-adenylated oligonucleotide to bind the 3′ end of a nucleic acid sequence from the sample to form a ligation product comprising the nucleic acid sequence; and
d) retrieving the ligation product.

9. The method of claim 8, wherein the retrieving step is performed by gel electrophoresis.

10. The method of claim 8, wherein the nucleic acid sequence is selected from the group consisting of single stranded DNA, double stranded DNA, single stranded RNA, double stranded RNA and a DNA-RNA chimera.

11. The method of claim 10, wherein the single stranded RNA is selected from the group consisting of microRNA, siRNA and snoRNA.

12. A method for amplifying a nucleic acid sequence from a sample comprising the steps of:
a) providing the adenylated oligonucleotide of claim 1;
b) contacting the adenylated oligonucleotide to a sample in the presence of ligase and in the absence of ATP;
c) allowing the adenylated oligonucleotide to bind the 3′ end of the nucleic acid sequence from the sample to form a first ligation product;
d) providing a second oligonucleotide sequence to the sample in the presence of ligase and ATP;
e) allowing the second oligonucleotide sequence to bind the first ligation product to form a second ligation product; and
f) amplifying the second ligation product.

13. A method for sequencing a plurality of nucleic acid sequences comprising the steps of:
a) providing a first oligonucleotide having a 3′ block and a 5′ phosphate;
b) providing a second oligonucleotide that is partially complementary to the first oligonucleotide;
c) allowing the first oligonucleotide and the second oligonucleotide to hybridize to form a duplex, wherein the second oligonucleotide has a 3′ overhang;
d) contacting the duplex with a DNA ligase and ATP;
e) allowing the ligase to adenylate the first oligonucleotide to form a pre-adenylated oligonucleotide;
f) contacting the adenylated oligonucleotide to a sample in the presence of ligase and in the absence of ATP;
g) allowing the adenylated oligonucleotide to bind the 3′ end of the nucleic acid sequence from the sample to form a first ligation product;
h) providing a third oligonucleotide sequence to the sample in the presence of ligase and ATP;
i) allowing the third oligonucleotide sequence to bind the first ligation product to form a second ligation product;
j) repeating steps a)-i) until a plurality of second ligation products are obtained; and
k) sequencing the plurality of second ligation products.
